(19) United States
(12) Patent Application Publication
(10) Pub. No.: US 2026/0151052 A1
(43) Pub. Date: Jun. 4, 2026

(54) Real-Time Determination of Biological Data Based on Images

(71) Applicant: Korro AI Limited, London (GB)

(72) Inventors: Philipp HULSDUNK, Berlin (DE); Markus SCHLATTMANN, Griesheim (DE); Matania Zvi KOCHAVI, Caesarea (IL); Maya KOCHAVI, New-York, NY (US)

(21) Appl. No.: 18/965,945

(22) Filed: Dec. 2, 2024

(51) Int. Cl. A61B 5/11; A61B 5/00; A61B 5/0205; A61B 5/024; A61B 5/113

(52) U.S. Cl. CPC A61B 5/1128; A61B 5/0205; A61B 5/113; A61B 5/7267; A61B 5/0077; A61B 5/02405; A61B 2576/02

(57) ABSTRACT

There are provided systems and methods comprising obtaining a sequence of images of a user, the sequence of images comprising a first image and a second image, determining data D_{optical_flow_torso} informative of a motion of at least part of a torso of the user between the first image and the second image, and feeding the data D_{optical_flow_torso}, or data derived from the data D_{optical_flow_torso}, to at least one machine learning model, to obtain biological data of the user.

### TECHNICAL FIELD

The presently disclosed subject matter relates, in general, to the field of image analysis, for determining biological data of a user, such as breathing data and/or heart rate.

### BACKGROUND

References considered to be relevant as background to the presently disclosed subject matter are listed below (acknowledgement of the references herein is not to be inferred as meaning that these are in any way relevant to the patentability of the presently disclosed subject matter):
Rehouma, Haythem, et al. “Advancements in methods and camera-based sensors for the quantification of respiration” Sensors 20.24 (2020): 7252;
Reyes, Bersain A., et al. “Tidal volume and instantaneous respiration rate estimation using a volumetric surrogate signal acquired via a smartphone camera”, IEEE journal of biomedical and health informatics 21.3 (2016): 764-777;
Nakajima, Kazuki, et al. “Evaluation of body motion by optical flow analysis”, Japanese journal of applied physics 36.5R (1997): 2929;
Nakajima, Kazuki, Yoshiaki Matsumoto, and Toshiyo Tamura, “Development of real-time image sequence analysis for evaluating posture change and respiratory rate of a subject in bed”, Physiological Measurement 22.3 (2001): N21;
Brieva, Jorge, Hiram Ponce, and Ernesto Moya-Albor. “Non-contact breathing rate monitoring system using a magnification technique and convolutional networks”, 15th international symposium on medical information processing and analysis, Vol. 11330. SPIE, 2020;
Guo, Tianqi, Qian Lin, and Jan Allebach. “Remote estimation of respiration rate by optical flow using convolutional neural networks”, Electronic Imaging 33 (2021): 1-11;
Clinic, Cleveland. Current Clinical Medicine: Expert Consult Premium Edition—Enhanced Online Features and Print. 2nd ed., W B Saunders, 2009.
Hassan, Mohamed Abul, et al. “Video-based heartbeat rate measuring method using ballistocardiography”, IEEE Sensors Journal 17.14 (2017): 4544-4557;
Kumar, Mayank, Ashok Veeraraghavan, and Ashutosh Sabharwal. “DistancePPG: Robust non-contact vital signs monitoring using a camera”, Biomedical optics express 6.5 (2015): 1565-1588;
Alinovi, Davide, et al. “Spatio-temporal video processing for respiratory rate estimation.” 2015 IEEE International Symposium on Medical Measurements and Applications (MeMeA) Proceedings. IEEE, 2015; and
JP6472086B2.

### GENERAL DESCRIPTION

In accordance with certain aspects of the presently disclosed subject matter, there is provided a system comprising one or more processing circuitries configured to obtain a sequence of images of a user, the sequence of images comprising a first image and a second image, determine data D_{optical_flow_torso} informative of a motion of at least part of a torso of the user between the first image and the second image, and feed the data D_{optical_flow_torso}, or data derived from the data D_{optical_flow_torso}, to at least one machine learning model, to obtain data D_{breathing} informative of the user's breathing.

In addition to the above features, the system according to this aspect of the presently disclosed subject matter can optionally comprise one or more of features (i) to (xxx) below, in any technically possible combination or permutation:
i. determining data D_{optical_flow_torso} comprises determining an optical flow field between at least part of the first image and at least part of the second image;
ii. determining data Doptical_flow_torso comprises determining a first optical flow field between at least part of the first image and at least part of the second image, and determining a second optical flow field between at least part of the first image and at least part of another image of the sequence, different from the first and second images;
iii. the at least one machine learning model comprises a temporal machine learning model;
iv. the data D_{breathing} is informative of breathing volume of the user;
V. the data D_{breathing} is informative of breathing strength of the user;
vi. the data D_{breathing} is informative of breathing rate of the user;
vii. the data D_{breathing} is informative of breathing phase of the user;
viii. the data D_{breathing} is informative of breathing volume of the user in the first image or in the second image, expressed relative to maximal breathing volume and/or minimal breathing volume within a period of time including the first image or the second image;
ix. the data D_{breathing} is informative of breathing volume of the user, expressed relative to maximal breathing volume and/or minimal breathing volume of the user;
x. the data D_{breathing} is informative of an absolute value of the breathing volume of the user;
xi. the system is configured to normalize the data D_{optical_flow_torso} into normalized data D_{optical_flow_torso_normalized} and feed the data D_{optical_flow_torso_normalized} to the at least one machine learning model, to obtain the data D_{breathing} informative of the user's breathing;
xii. normalizing the data D_{optical_flow_torso} into the data D_{optical_flow_torso_normalized} comprises performing at least one of: (a) a normalization of the data D_{optical_flow_torso} with respect to at least one of: one or more dimensions of the user, or an orientation of the user; or (b) a normalization of the data D_{optical_flow_torso} with respect to time;
xiii. normalizing the data D_{optical_flow_torso} into the data D_{optical_flow_torso_normalized} comprises normalizing the data D_{optical_flow_torso} with respect to at least one of one or more dimensions of the user in one or more images of the sequence of images, or an orientation of the user in one or more images of the sequence of images;
xiv. the data D_{optical_flow_torso} comprises an optical flow field between the first image acquired at time t₁, and the second image acquired at time t₂, wherein said normalizing of the data D_{optical_flow_torso} into the data D_{optical_flow_torso_normalized} is based on a ratio between the optical flow field and a time interval between t₁ and t₂;
xv. the system is configured to generate data D_{optical_flow_torso_normalized} based on a transformation of the data D_{optical_flow_torso} into an area of fixed size, and feed the data D_{optical_flow_torso_normalized} to the at least one machine learning model, to obtain the data D_{breathing} informative of the user's breathing;
xvi. said transformation enables converting predefined points of the torso of the user into predefined edges of the area of fixed size;
xvii. the system is configured to use at least part of the sequence of images acquired by an acquisition device to determine data Dₚₒₛₑ informative of body nodes of the user, and use the data Dₚₒₛₑ to perform at least one of (a) or (b): (a) detecting whether the distance of the user from the acquisition device is above a first threshold; (b) detecting whether an amplitude or a speed of a motion of the user is above a second threshold;
xviii. the first image and the second image have been acquired by an acquisition device, wherein the first image and the second image have not been acquired consecutively by the acquisition device;
xix. the data D_{optical_flow_torso} is informative of three-dimensional optical flow between at least part of the torso of the user in the first image and at least part of the torso of the user in the second image, wherein the system is configured to generate data D_{optical_flow_torso_normalized} based on a transformation of the data D_{optical_flow_torso} into a volume of fixed size, and feed the data D_{optical_flow_torso_normalized} to the at least one machine learning model, to obtain the data D_{breathing} informative of the user's breathing;
xx. the system is configured to obtain the data D_{breathing} informative of the user's breathing without requiring the user to take part to a calibration process;
xxi. the system is configured to determine the data D_{breathing} informative of the user's breathing in a scenario in which a position of the user is moving between the first image and the second image;
xxii. the system is configured to obtain data informative of the user's breathing for at least one given image of the sequence, even if the user was absent in one or more images of the sequence preceding this given image, without requiring the user to take part to a calibration process;
xxiii. the system is configured to detecting whether a frequency of change of the data D_{optical_flow_torso} in a plurality of images of the sequence is above a threshold;
xxiv. the system is configured to normalize the data D_{optical_flow_torso} into normalized data D_{optical_flow_torso_normalized} and detecting whether a frequency of change of the data D_{optical_flow_torso_normalized} in a plurality of images of the sequence is above a threshold;
xxv. the system is configured to use the data D_{optical_flow_torso} to detect one or more occlusions between the user and an acquisition device which has acquired the sequence of images; and
xxvi. the system is configured to normalize the data D_{optical_flow_torso} into normalized data D_{optical_flow_torso}_normalize and use the data D_{optical_flow_torso_normalized} to detect one or more occlusions between the user and an acquisition device which has acquired the sequence of images;
xxvii. the system is configured to feed the data D_{optical_flow_torso}, or the data derived therefrom, to the at least one machine learning model, to obtain data informative of the user's heart rate;
xxviii. the data informative of the user's heart rate includes at least one of the user's heart rate, or the user's heart rate variability; and
xxix. the system is configured to obtain data informative of the user's heart rate, and feed the data D_{optical_flow_torso}, or data derived therefrom, and the data informative of the user's heart rate to the at least one machine learning model, to obtain data D_{breathing} informative of the user's breathing; and
xxx. the system is configured to obtain the data D_{breathing} informative of the user's breathing, feed the data informative of the user's heart rate, or data derived therefrom, and the data D_{breathing} informative of the user's breathing to another machine learning model, to obtain at least one of updated data informative of the user's heart rate, or updated data D_{breathing} informative of the user's breathing.

According to another aspect of the presently disclosed subject matter there is provided a non-transitory computer readable medium comprising instructions that, when executed by one or more processing circuitries, cause the one or more processing circuitries to perform: obtaining a sequence of images of a user, the sequence of images comprising a first image and a second image, determining data D_{optical_flow_torso} informative of a motion of at least part of a torso of the user between the first image and the second image, and feeding the data D_{optical_flow_torso}, or data derived from the data D_{optical_flow_torso}, to at least one machine learning model, to obtain data D_{breathing} informative of the user's breathing.

In addition to the above features, the non-transitory computer readable medium comprises instructions that, when executed by the one or more processing circuitries, cause the one or more processing circuitries to perform or to include one or more of features (i) to (xxx) above, in any technically possible combination or permutation.

According to another aspect of the presently disclosed subject matter there is provided a method comprising (by one or more processing circuitries) obtaining a sequence of images of a user, the sequence of images comprising a first image and a second image, determining data D_{optical_flow_torso} informative of a motion of at least part of a torso of the user between the first image and the second image, and feeding the data D_{optical_flow_torso}, or data derived from the data D_{optical_flow_torso}, to at least one machine learning model, to obtain data D_{breathing} informative of the user's breathing.

In addition to the above features, the method can include one or more of features (i) to (xxx) above, in any technically possible combination or permutation.

According to another aspect of the presently disclosed subject matter there is provided a system comprising one or more processing circuitries configured to obtain a sequence of images of a user, the sequence of images comprising a first image and a second image, determine data D_{optical_flow_torso} informative of a motion of at least part of a torso of the user between the first image and the second image, and feed the data D_{optical_flow_torso}, or data derived from the data D_{optical_flow_torso}, to at least one machine learning model, to obtain data informative of the user's heart rate.

In addition to the above features, the system can include one or more of features (i) to (xxx) above, in any technically possible combination or permutation.

According to another aspect of the presently disclosed subject matter there is provided a non-transitory computer readable medium comprising instructions that, when executed by one or more processing circuitries, cause the one or more processing circuitries to perform: obtaining a sequence of images of a user, the sequence of images comprising a first image and a second image, determining data D_{optical_flow_torso} informative of a motion of at least part of a torso of the user between the first image and the second image, and feeding the data D_{optical_flow_torso}, or data derived from the data D_{optical_flow_torso}, to at least one machine learning model, to obtain data informative of the user's heart rate.

In addition to the above features, the non-transitory computer readable medium comprises instructions that, when executed by the one or more processing circuitries, cause the one or more processing circuitries to perform or to include one or more of features (i) to (xxx) above, in any technically possible combination or permutation.

According to another aspect of the presently disclosed subject matter there is provided a method comprising (by one or more processing circuitries) obtaining a sequence of images of a user, the sequence of images comprising a first image and a second image, determining data D_{optical_flow_torso} informative of a motion of at least part of a torso of the user between the first image and the second image, and feeding the data D_{optical_flow_torso}, or data derived from the data D_{optical_flow_torso}, to at least one machine learning model, to obtain data informative of the user's heart rate.

In addition to the above features, the method can include one or more of features (i) to (xxx) above, in any technically possible combination or permutation.

In accordance with certain aspects of the presently disclosed subject matter, there is provided a system comprising one or more processing circuitries configured to obtain a sequence of images of a user, the sequence of images comprising a first image and a second image, determine data D_{optical_flow_torso} informative of a motion of at least part of a torso of the user between the first image and the second image, and feed the data D_{optical_flow_torso}, or data derived from the data D_{optical_flow_torso}, to at least one machine learning model, to obtain biological data of the user. The biological data can include e.g., data D_{breathing} informative of the user's breathing and/or data informative of the user's heart rate.

In addition to the above features, the system can include one or more of features (i) to (xxx) above, in any technically possible combination or permutation. Corresponding method (executing the features mentioned with respect to the system) and non-transitory computer readable medium comprising instructions that, when executed by one or more processing circuitries, cause the one or more processing circuitries to perform this method are also provided.

According to some examples, the proposed solution enables determining breathing data and/or heart rate based on images of a user, without requiring lengthy or complex calibration.

According to some examples, the proposed solution is invariant or quasi-invariant to the user's orientation and/or scale.

According to some examples, the proposed solution enables auto-scaling while the user is moving.

According to some examples, the proposed solution enables determination of data informative of the breathing volume with no need of prior knowledge in breathing exercises.

According to some examples, the proposed solution enables determining breathing data based on images of a user, without requiring the user to stand, sit or lie still in front of the camera.

According to some examples, the proposed solution enables determining breathing data and/or heart rate based on images of a user, in various types of activities performed by the user.

According to some examples, the proposed solution enables determining breathing data and/or heart rate based on images of a user, even if the user exits the field of view of the camera and then returns to the field of view of the camera, without requiring additional calibration.

According to some examples, the proposed solution enables determining in real-time breathing data and/or heart rate based on images of a user.

According to some examples, the proposed solution provides an advanced experience to users, e.g. on mobile devices.

According to some examples, the proposed solution is usable in various applications, such as applications including behavioral health, meditation, and various kinds of regulation activities.

According to some embodiments, the proposed solution can be used on various device types (e.g. mobile, laptops, smart mirrors, etc.).

### BRIEF DESCRIPTION OF THE DRAWINGS

In order to understand the disclosure and to see how it may be carried out in practice, embodiments will now be described, by way of non-limiting example only, with reference to the accompanying drawings, in which:

FIG. 1A illustrates a generalized block diagram of a system which can be used to perform various methods as described hereinafter;

FIG. 1B illustrates a variant of the system of FIG. 1A;

FIG. 2 illustrates a generalized flow-chart of a method of determining data informative of the user's breathing, according to some examples of the invention;

FIG. 3 illustrates a non-limitative example of an optical flow vector between two images;

FIG. 4 illustrates a non-limitative example of an optical flow field between two images;

FIG. 5 illustrates another non-limitative example of an optical flow field between two images;

FIG. 6 illustrates a non-limitative example of a sequence of images (frames) acquired by an acquisition device;

FIG. 7 illustrates a non-limitative example of an image of the torso of a user, on which various output data have been superimposed, such as breathing data;

FIG. 8 illustrates a non-limitative example of a scale used to represent the user's breathing volume;

FIG. 9 illustrates a generalized flow-chart of a method of pre-processing a sequence of images of a user, acquired by an acquisition device;

FIG. 10 illustrates a non-limitative example of dimensions of the torso of a user in an image;

FIG. 11 illustrates a generalized flow-chart of a method of determining data informative of the user's breathing, according to some examples of the invention;

FIG. 12 illustrates a generalized flow-chart of a method of normalizing optical flow data, according to some examples of the invention;

FIG. 13 illustrates a generalized flow-chart of a method of normalizing optical flow data, according to some examples of the invention;

FIG. 14 illustrates a non-limitative example of the method of FIG. 13;

FIG. 15 illustrates a generalized flow-chart of a method of normalizing optical flow data, according to some examples of the invention;

FIG. 16 illustrates a non-limitative example of the method of FIG. 15;

FIG. 17 illustrates a generalized flow-chart of a method of normalizing optical flow data, according to some examples of the invention;

FIGS. 18A and 18B illustrate non-limitative examples of the method of FIG. 17;

FIG. 19 illustrates a non-limitative example of a projection used in the method of FIG. 17;

FIG. 20 illustrates a generalized flow-chart of a method of normalizing optical flow data, according to some examples of the invention;

FIG. 21 illustrates a generalized flow-chart of a method of detecting noisy or dark images, according to some examples of the invention;

FIG. 22 illustrates a generalized flow-chart of a method of detecting occlusions, according to some examples of the invention;

FIG. 23 illustrates a generalized flow-chart of a method of detecting abnormal optical flow vectors, according to some examples of the invention;

FIG. 24 illustrates a generalized flow-chart of a method of determining a breathing ratio, according to some examples of the invention;

FIG. 25 illustrates a generalized flow-chart of a method of determining data informative of the user's breathing, according to some examples of the invention;

FIG. 26 illustrates a generalized flow-chart of a method of determining data informative of the user's heart rate, according to some examples of the invention;

FIG. 27 illustrates a generalized flow-chart of a method of training a machine learning model to determine data informative of the user's breathing, according to some examples of the invention; and

FIG. 28 illustrates a generalized flow-chart of a method of training a machine learning model to determine data informative of the user's heart rate, according to some examples of the invention.

### DETAILED DESCRIPTION OF EMBODIMENTS

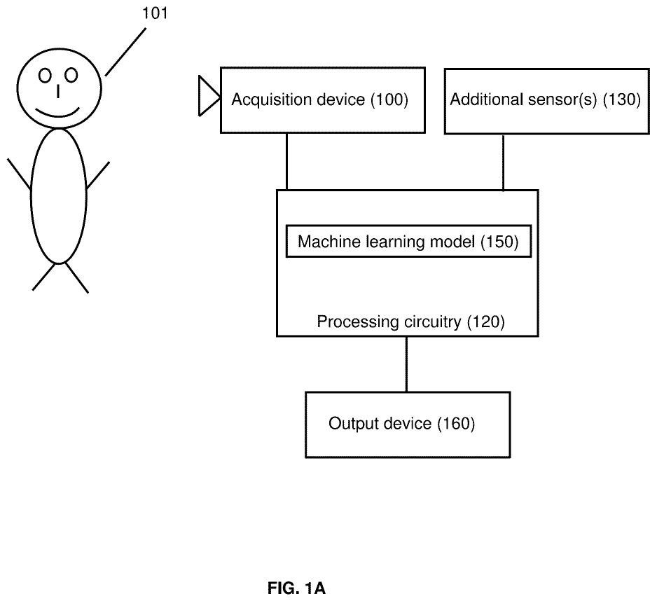

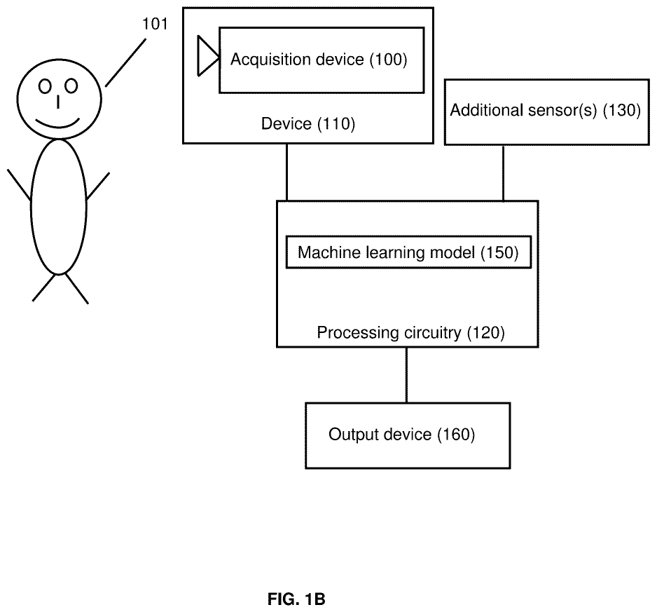

Attention is drawn to FIG. 1A. As visible in FIG. 1A, an acquisition device 100 is configured to acquire a sequence of images of a user 101 (or of a plurality of users). In some examples, the sequence of images is acquired while the user is performing an activity. Non-limitative examples of activities include a game, a sport and/or fitness activity, a medical test, etc.

The acquisition device 100 can correspond to a camera (e.g., video camera), which acquires the sequence of images over time. In some examples, the acquisition device 100 corresponds to a stand-alone camera. In other examples, the acquisition device 100 is part of a device 110 (see FIG. 1B), such as a smartphone, a laptop, or an augmented reality (AR) headset. This list is not limitative. Note that the acquisition device 100 can be either fixed, mobile, or both fixed and mobile (depending on the time period).

In some examples, a plurality of acquisition devices 100 can be used. The field of view of the plurality of acquisition devices 100 can partially or fully overlap. In this case, a plurality of sequences of images of the user(s) can be obtained.

The sequence of images acquired by the one or more acquisition device(s) 100 is transmitted to one or more processing circuitries 120, using wire or wireless communication (e.g., Wi-Fi, Internet, Bluetooth, etc.). As explained hereinafter, the one or more processing circuitries 120 are operative to process the sequence of images in order to determine biological data (also called biological indicators/parameters) of the user 101, such as data informative of the user's breathing, and/or data informative of the user's heart.

Note that the one or more processing circuitries 120 can be part of the device 110, or can be external to it. In some examples, the one or more processing circuitries 120 can be located at a remote location (e.g., cloud). In some examples, some of the processing operations are performed locally, and some of the processing operations are performed at a remote location.

In some examples, one or more additional sensor(s) 130 can be used. A non-limitative example of additional sensors includes a heart sensor, enabling determining the heart rate of the user, such as (but not limited to) a spirometer, etc. Data collected by the additional sensor(s) can be transmitted to the one or more processing circuitries 120, using wire or wireless communication.

As visible in FIGS. 1A and 1B, the one or more processing circuitries 120 implement at least one machine learning model 150. Note that the at least one machine learning model 150 can include a plurality of machine learning models (sub-networks) that are connected (e.g., in series).

In some examples, the machine learning model 150 is a deep neural network (DNN). By way of non-limiting example, the layers of the machine learning model 150 can be organized in accordance with Convolutional Neural Network (CNN) architecture, such as a fully Convolutional Neural Network (CNN), with Recurrent Neural Network architecture, Generative Adversarial Network (GAN) architecture, or otherwise. This is not limitative. In some examples, the machine learning model 150 includes a temporal machine learning model, such as LSTM (Long short-term memory), WaveNet, Attention Models, etc. A temporal machine learning model is a model that takes into account time and/or history of data.

In some examples, the machine learning model 150 includes a Convolutional Neural Network, used to reduce the dimensions of the input data, followed by a temporal machine learning model, such as a LSTM (Long short-term memory) or another temporal machine learning model. Other architectures can be used and this is not limitative.

Each layer of the machine learning model 150 can include multiple basic computational elements (CE), typically referred to in the art as dimensions, neurons, or nodes. Generally, computational elements of a given layer can be connected with CEs of a preceding layer and/or a subsequent layer. Each connection between a CE of a preceding layer and a CE of a subsequent layer is associated with a weighting value. A given CE can receive inputs from CEs of a previous layer via the respective connections, each given connection being associated with a weighting value which can be applied to the input of the given connection. The weighting values can determine the relative strength of the connections and thus the relative influence of the respective inputs on the output of the given CE. The given CE can be configured to compute an activation value (e.g., the weighted sum of the inputs) and further derive an output by applying an activation function to the computed activation. The activation function can be, for example, an identity function, a deterministic function (e.g., linear, sigmoid, threshold, or the like), a stochastic function, or other suitable function. The output from the given CE can be transmitted to CEs of a subsequent layer via the respective connections. Likewise, as above, each connection at the output of a CE can be associated with a weighting value which can be applied to the output of the CE prior to being received as an input of a CE of a subsequent layer. Further to the weighting values, there can be threshold values (including limiting functions) associated with the connections and CEs. The weighting and/or threshold values of the machine learning model 150 can be initially selected prior to training, and can be further iteratively adjusted or modified during training to achieve an optimal set of weighting and/or threshold values in a trained machine learning model 150. After each iteration, a difference (also called loss function) can be determined between the actual output produced by the machine learning model 150 and the target output associated with the respective training set of data. The difference can be referred to as an error value. Training can be determined to be complete when a cost or loss function indicative of the error value is less than a predetermined value, or when a limited change in performance between iterations is achieved.

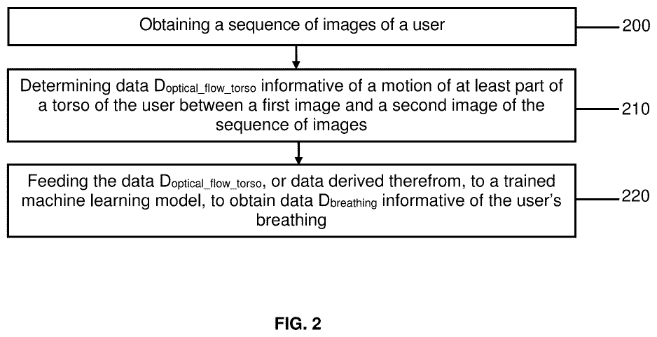

The one or more processing circuitries 120 can be used to execute one or more of the methods described hereinafter (see e.g., FIGS. 2, 9, 11, 12, 13, 15, 17, 20 to 28).

An output device 160 (also called display device, such as a screen) can be used to output the results of the processing performed by the processing circuitry 120. In some examples, the output device 160 can output both the sequence of images acquired by the acquisition device 100 and the results of the processing performed by the processing circuitry 120. In the example of FIG. 1B, the output device 160 can be part of the device 110 (for example, it can correspond to the screen of the device), or can be external to it (an additional screen). In some examples, the results of the processing can be output as an audio feedback (generated by a loudspeaker). In some examples, the results of the processing can be output both as a visual and audio feedback.

Attention is now drawn to FIG. 2, which describes a method enabling determining biological data of a user, such as breathing data.

The method of FIG. 2 includes obtaining (operation 200) a sequence of images (also called frames) of a user. The sequence of images has been acquired e.g., by the acquisition device 100. Note that one or more of the images of the sequence can include multiple users. In this case, it can be defined that the user who is the closest to the acquisition device 100 is the user whom biological data needs to be extracted. Detection of the closest user can be performed by extracting the pose of the different users and using the extracted pose of the users to determine the dimensions of the body of the users in the image, and using these dimensions to identify the closest user. This is not limitative.

In other examples, a tracking of a given user can be performed based on features (e.g., face and/or clothes) specific to this given user. This tracking enables extracting biological data for this given user, and not for other users. Note that it is possible to extract biological data of multiple users from the sequence of images, by tracking each user separately, and performing the various methods described hereinafter for each user.

One or more of the images of the sequence can include additional elements which differ from the user, such as elements (walls, furniture, etc.) of the environment in which the user is located.

The sequence of images can be pre-processed. This pre-processing enables detecting images of the sequence which cannot be used to accurately extract biological data of the user. This can be due to various factors, such as the presence of occlusions, insufficient signal to noise ratio (due e.g., to low lightness/darkness of the images), very large distance of the user to the acquisition device, very much motion of the user, etc. This will be described hereinafter.

Assume that a first image (acquired at time t) of the sequence of images and a second image (acquired at time t′, posterior to time t) of the sequence of images are selected from the sequence of images. A first area of the first image is informative of the torso (or at least part of it) of the user, at time t. A second area of the second image is informative of the torso (or at least part of it) of the user, at time t.

The method of FIG. 2 further includes determining (operation 210) data D_{optical_flow_torso} informative of a motion of at least part of a torso of the user between the first image and the second image. The motion (also called apparent motion) of the torso includes e.g. the displacement and/or the velocity of the torso between the first image and the second image.

In particular, operation 210 can include determining optical flow between at least part of the first image and at least part of the second image. Optical flow or optic flow corresponds to the pattern of apparent motion of object(s) in a visual scene caused by the motion of the object(s) relative to an observer (in this case, the acquisition device 100).

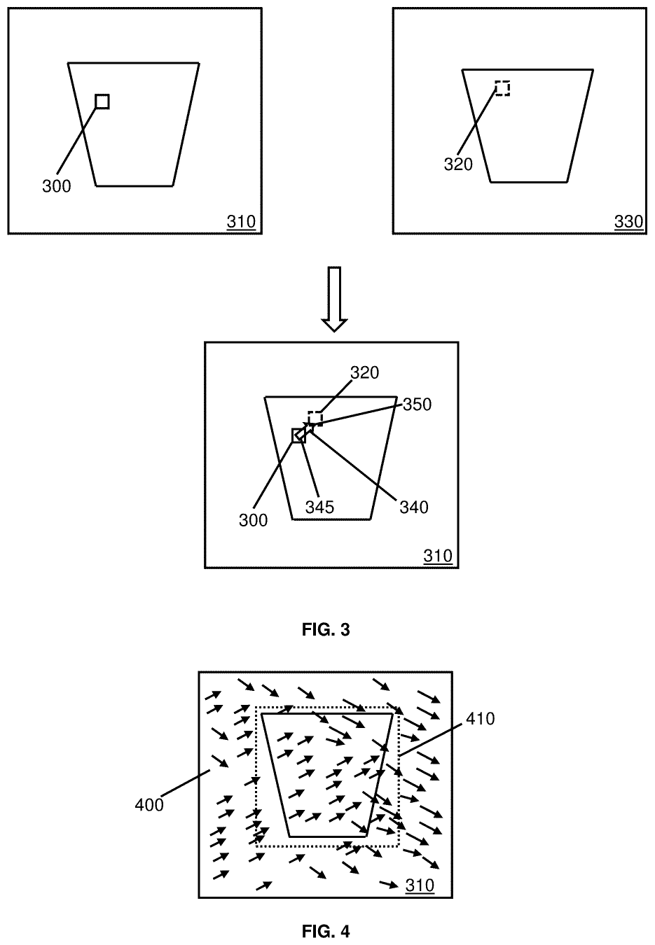

Optical flow determination can include determining, for each pixel (see e.g., pixel 300, also called voxel 300, in FIG. 3, located at position (x,y) at time t in the first image 310), the position (x′,y′) of this pixel (see e.g., pixel 320 in FIG. 3, corresponding to the same pixel with a different position due to its relative motion with respect to the acquisition device 100) at time t′ in the second image 330. A vector 340 can be generated: the first extremity (tail) 345 of the vector 340 corresponds to the position (x,y) of the pixel at time t, and the second extremity 350 of the vector (tip of the arrow) corresponds to the position (x′,y′) of the same pixel at time t′. In the bottom part of FIG. 3, the vector 340 has been superimposed on the first image 310, in order to show the relative motion of the pixel 300 between time t and time t′. Note that the optical flow generated for an image can be computed between this image and another image acquired afterwards, or an image acquired beforehand.

Determination of the optical flow between the first image and the second image can be performed using e.g., neural networks. An example of neural network that can be used includes Recurrent All-Pairs Field Transforms (RAFT). This example is not limitative.

Note that if the acquisition device 100 is mobile, the motion of the acquisition device 100 should be cancelled in order to determine the optical flow of the torso of the user. This can be performed based on measurements of one or more sensors (one or more position/velocity sensors, which determine motion of the acquisition device 100, can be used—the position/velocity sensor(s) can be coupled to the acquisition device 100, or to the device 110 embedding the acquisition device 100) can be used, and/or based on finding a major direction of optical movement within the camera image (this enables differentiating between the motion due to the acquisition device 100 and the motion due to the torso).

In some examples (see FIG. 4), it is possible to perform the optical flow determination on the whole or most of the first image (or in an equivalent manner in the whole second image). A raw field 400 of optical flow vectors is therefore obtained. It is then possible to select, within the raw field of optical flow vectors, an area 410 informative of the torso of the user. The area 410 can be selected to encompass the torso of the user. This enables obtaining the data D_{optical_flow_torso} (informative of the motion of at least part of the torso of the user between the first image and the second image). In particular, the data D_{optical_flow_torso} can include the optical flow vectors located in the area 410.

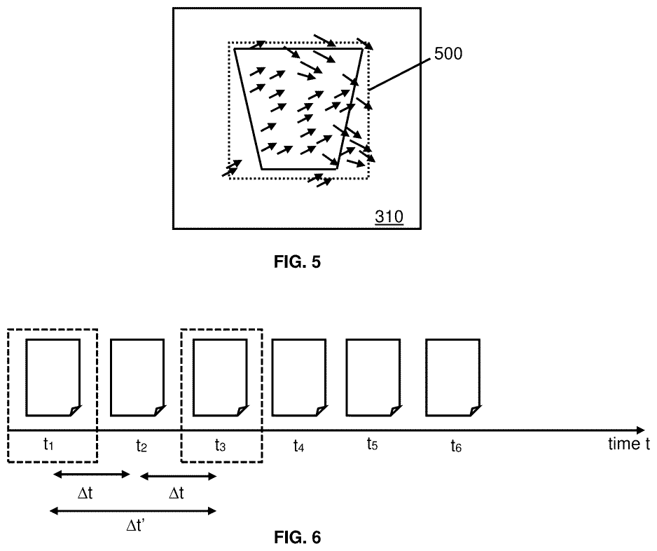

In some other examples (see FIG. 5), it is possible to perform the optical flow determination on a specific area 500 of the first image (or in an equivalent manner on a specific area of the second image). This specific area 500 is selected to be informative of the torso of the user. In particular, this specific area 500 can be selected to encompass the torso of the user. For each pixel of this specific area 500, the corresponding position of the pixel in the second image is determined. This enables obtaining the data D_{optical_flow_torso} (informative of the motion of at least part of the torso of the user between the first image and the second image), which includes the optical flow vectors of the specific area 500.

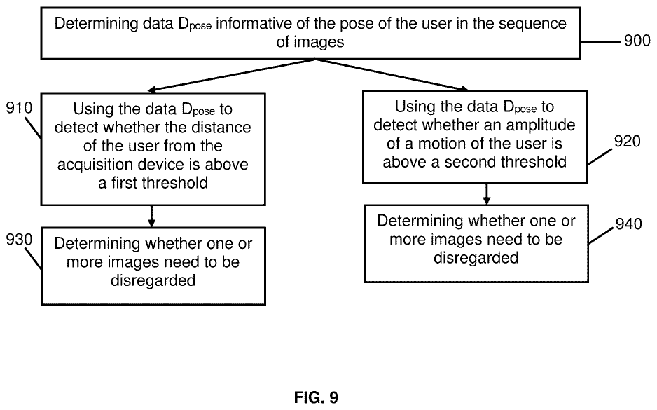

Note that determination of the position of the torso of the user in the images can be performed as explained hereinafter with reference to operation 900 in FIG. 9.

As visible in FIG. 6, the sequence of images includes a succession of images (frames) acquired at different time instants t₁, t₂, t₃, . . . , t₆, etc. The images are acquired at a certain acquisition rate (frequency rate, corresponding to a certain time rate Δt), which depends e.g., on the acquisition device 100. In order to increase the amplitude of motion of the user's torso between two images (thereby obtaining optical flow data D_{optical_flow_torso} which are more exploitable), it is possible to select a first image and a second image which have not been acquired consecutively by the acquisition device 100. In other words, the time interval Δt′ between the first image and the second image (used to determine the optical flow) is selected to be greater than the time interval Δt between two consecutive images acquired by the acquisition device 100 according to its acquisition rate. A non-limitative example of the time interval Δt′ is ½s, or more.

In some examples, it is possible to determine (as part of the data D_{optical_flow_torso}) the optical flow between the first image and a plurality of different images of the sequence, acquired at different instants of time. In other words, the optical flow is calculated for different time intervals. For example, a first optical flow field can be determined between the first image I₁ (acquired at time t₁) and another image Iⱼ (acquired at time tⱼ, with j>1—this corresponds to a time interval tⱼ−t₁), a second optical flow field can be determined between the first image I₁ (acquired at time t₁) and another image Iₖ (acquired at time tₖ, with k>j—this corresponds to a time interval tₖ−t₁), etc. Note that the “first image” I₁ is not necessarily the first image of the sequence and can correspond to another image of the sequence.

In some examples, the time interval which is selected to determine the optical flow field(s) can be dynamic or variable. For example (this is not limitative), for each image of the sequence for which the optical flow field is computed (as part of the data D_{optical_flow_torso}), the optical flow field is computed with respect to another image in which the user has completely exhaled, and/or with respect to another image in which the user has completely inhaled. Note that the images in which the user has completely exhaled, or completely inhaled, can be identified by the machine learning model 150, as explained hereinafter.

In some examples, the D_{optical_flow_torso} (which is fed to the machine learning model 150) contains (for at least one image of the sequence, or for multiple images of the sequence) both optical flow field(s) determined with a fixed time interval, and optical flow field determined with a variable time interval.

As explained with reference to FIGS. 1A and 1B, a machine learning model 150 (trained machine learning model) is usable to determine biological data of the user. Training of this machine learning model 150 is discussed hereinafter.

In some examples, the method of FIG. 2 further includes feeding (operation 230) the data D_{optical_flow_torso} to the machine learning model 150. As mentioned above, the D_{optical_flow_torso} can include, for a given image of the sequence, an optical flow field, or multiple optical fields. In some examples, the method of FIG. 2 includes feeding (operation 230) data derived from the data D_{optical_flow_torso} to the machine learning model 150. In particular, as explained hereinafter, the data D_{optical_flow_torso} can be normalized into normalized data D_{optical_flow_torso_normalized}. As explained hereinafter, this normalization can correspond to a spatial normalization (e.g., with respect to dimension(s) and/or orientation of the user) and/or to a temporal normalization (with respect to time). The normalized data D_{optical_flow_torso_normalized}, which have been derived from the data D_{optical_flow_torso}, can be fed to the machine learning model 150. In some examples, both the data D_{optical_flow_torso} and the data D_{optical_flow_torso_normalized} can be fed to the machine learning model 150.

This enables obtaining data D_{breathing} informative of the user's breathing. In some examples, the output of the machine learning model 150 corresponds to the data D_{breathing}. In some examples, the output of the machine learning model 150 is further processed to obtain the data D_{breathing}.

Note that the method of FIG. 2 can be performed in real time or quasi time, while the sequence of images is acquired by the acquisition device 100. In addition, the method of FIG. 2 can be performed repeatedly for different images of the sequence. For each given image of the sequence, the corresponding optical flow is computed with respect to another image acquired after the given image. Note that it is possible to compute a plurality of optical flow fields for a given image, e.g., by computing optical flows between the given image and different other images of the sequence. The corresponding optical flow field(s), or data derived thereof (normalized optical flow(s)) is/are then fed to the machine learning model 150 to generate the data D_{breathing} for this given image. In other words, for each image of the sequence acquired at a given time t, the corresponding data D_{breathing} informative of the user's breathing at time t is computed.

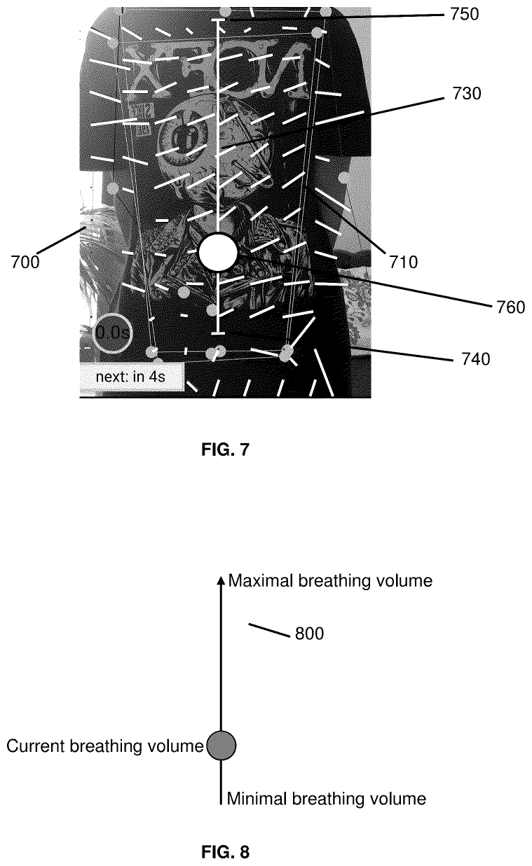

In some examples, the data D_{breathing} can be output, such as on the display device 160. In particular, for each image for which the data D_{breathing} has been computed, the data D_{breathing} can be displayed together with the image. The data D_{breathing} can be superimposed on the image, and/or can be used to create visual elements indicative of D_{breathing}. In some examples, the data D_{optical_flow_torso} or the data D_{optical_flow_torso_normalized} can be also displayed, e.g. on the image for which the data D_{optical_flow_torso} or D_{optical_flow_torso_normalized} has been computed. A non-limitative example is provided in FIG. 7, in which an image 700 of the torso of the user is displayed. The optical flow (D_{optical_flow_torso}) 710 of the area of the image 700 informative of the torso of the user is also displayed. Data informative of the breathing volume of the user is also displayed on the image 700. In the non-limitative example of FIG. 7, a scale 730 is displayed on the image of the user. The scale 730 includes a first indicator 740 indicative of the minimal breathing volume and a second indicator 750 indicating of maximal breathing volume. A moving element 760 (e.g., a disk) moves along the scale 730 between the first indicator 740 and the second indicator 750. The position of the moving element 760 along the scale 730 depends on the breathing volume determined for the image. This provides intuitive feedback to the user.

In some examples, the data D_{breathing} is informative of the breathing volume of the user. The breathing volume output by the machine learning model 150 is an estimate (approximation). In some examples, this breathing volume corresponds to a relative breathing volume. It can be defined according to a certain scale (see scale 800 in FIG. 8), which defines the maximal breathing volume and the minimal breathing volume. This scale can define the breathing volume according to a scale between 0% (minimal breathing volume) to 100% (maximal breathing volume). This breathing volume can be a relative volume since the actual vital capacity (volume of air breathed out after the deepest inhalation) is unknown.

In some examples, in the training stage, certain training data are annotated as corresponding to the maximal breathing volume, and other training data are annotated as corresponding to the minimal breathing volume, etc. Therefore, the trained machine learning model 150 is operative to output the breathing volume or other data informative of the user's breathing. This is not limitative and other training methods can be used, in which different annotations of the training data are used. For each image that has been processed as explained in the various methods described herein, the corresponding breathing volume (as expressed according to the scale 800) can be output.

Note that different conventions can be used to define the breathing volume. In some examples, the breathing volume can refer to the volume of air within the lungs. In this case, when the user inhales, the breathing volume increases, and, when the user exhales, the breathing volume decreases. Alternatively, the breathing volume can refer to the volume of air expired by the user. In this case, when the user exhales, the breathing volume increases, and when the user inhales, the breathing volume decreases.

In some examples, the breathing volume can correspond to an absolute value. This can be achieved if users have performed breathing measurements using medical devices, such as a spirometer. In this case, during the training phase, images of respective users are used and annotated with a percentage with respect to their real respective breathing volume, obtained e.g., from the spirometer. Examples of absolute breathing volume include e.g., the tidal volume, or the difference of volume between a full inspiration and a full exhalation. Note that it is possible to train the machine learning model 150 with training images and breathing data of users which differ from the user that has been acquired in the sequence of images. This is not limitative. During the prediction phase, the trained machine learning model 150 is able to determine, based on the input that it receives (D_{optical_flow_torso} and/or D_{optical_flow_torso_normalized}), the corresponding breathing volume.

In some examples, the machine learning model 150 can operate in different modes (different prediction modes). In a first mode, the machine learning model 150 provides a relative breathing volume (expressed e.g., between 0% and 100%) which is expressed with respect to a current time window (also called time chunk). Assume for example that the user is currently breathing, within a certain period of time, with a breathing volume varying between half of a full exhalation, and half of a full exhalation (these values are not limitative). The machine learning model 150 will assign to the breathing volume corresponding to half of a full inhalation and to half of a full exhalation the extremum values of the scale (0% and 100%). The other images in which the user is breathing between these two extreme values will get a breathing volume, which is scaled between 0% and 100%. Assume that the user changes his breathing mode, and is now currently breathing, within another period of time, with a breathing volume varying between a full exhalation, and a full exhalation (these values are not limitative). The machine learning model 150 will assign to the breathing volume corresponding to a full inhalation and to a full exhalation the extremum values of the scale (0% and 100%). The other images in which the user is breathing between these two extreme values will get a breathing volume, which is scaled between 0% and 100%. In other words, the machine learning model 150 adapts the scale of the breathing volume to the current breathing intensity of the user. In this first mode, the breathing volume output by the machine learning model 150 is expressed relative to the extremum phases of inhalation/exhalation of the user in the current period of time.

One advantage of this first mode is to be sensitive even to small amplitudes of the variation of the user's breathing. This is particularly beneficial to detect e.g., the rhythm of the user's breathing, or other applications in which a relative value of the breathing volume is more needed than an absolute of the breathing volume.

In a second mode, the machine learning model 150 expresses the breathing volume with respect to a scale in which the maximal breathing volume and the minimal breathing volume are assessed over e.g., the whole sequence of images (and not according to specific chunks of time as in the first mode). The machine learning model 150 can keep track internally, as a state, the maximal and minimal breathing volumes that it detects along the sequence of images and use them to define the scale according to which the breathing volume is output. If the user performs a full inhalation and a full exhalation during the activity, then the breathing volume will be expressed as a percentage of the maximal breathing volume of the user. Therefore, if the user has provided his maximal breathing volume (maximal lung volume) to the system, and/or the maximal breathing volume of this user has been measured using a device such as a spirometer, the absolute breathing volume can be output for each image, by multiplying the percentage provided by the machine learning model 150 with the maximal breathing volume.

In a third mode, for each current image of the user, at least two optical flow fields are fed to the machine learning model 150. A first optical flow field corresponds to the optical flow field between the current image and the image of the user corresponding to a full inhalation (in some examples, this image can be identified by asking the user to provide an indication to the system when he has reached a full inhalation). A second optical flow field corresponds to the optical flow field between the current image and the image of the user corresponding to a full exhalation (in some examples, this image can be identified by asking the user to provide an indication to the system when he has reached a full exhalation). The first and second optical flow fields are fed to the machine learning model 150 which outputs, for the current image, a percentage of the current breathing volume with respect to the maximal breathing volume. In this mode, the scale is not adaptive to the current breathing mode of the user, but is expressed with respect to the breathing volume during full exhalation and full inhalation. Then, if the user has provided his maximal breathing volume (maximal lung volume) to the system, and/or the maximal breathing volume of this user has been measured using a device such as a spirometer, the absolute breathing volume can be output for each image, by multiplying the percentage provided by the machine learning model 150 with the maximal breathing volume. Note that these three modes of operation are not limitative and other modes can be used.

Attention is now drawn to FIG. 9. As visible in FIG. 9, the images of the sequence can be processed (operation 900) in order to extract the pose (noted Dₚₒₛₑ) of the user from the images. Dₚₒₛₑ can include the position of the body nodes (e.g., hips, shoulders, head, etc.—also called body joints) of the user in the image. Non-limitative examples of algorithms that can be used to determine the pose of the user include BlazePose, YOLOv8, etc.

The position of the torso of the user can therefore be determined in the images, based on the extracted pose of the user. In some examples, the torso can be defined as the area of the body defined by four edges: the two shoulders and the two hips. This is however not limitative and other definitions can be used.

In some examples, operation 900 includes extracting the 3D pose of the user in the image. This provides the 3D position (e.g., in the referential of the camera) of various pixels informative of the user in the image. A non-limitative example of an algorithm that can be used to determine the 3D pose of the user includes the FinePOSE algorithm. The 3D position differs from the 2D position in that it further includes the depth of each pixel.

As visible in FIG. 9, the data Dₚₒₛₑ can be used to determine (operation 910) whether the distance of the user from the acquisition device 100 is above a certain threshold (first threshold) in one or more images of the sequence.

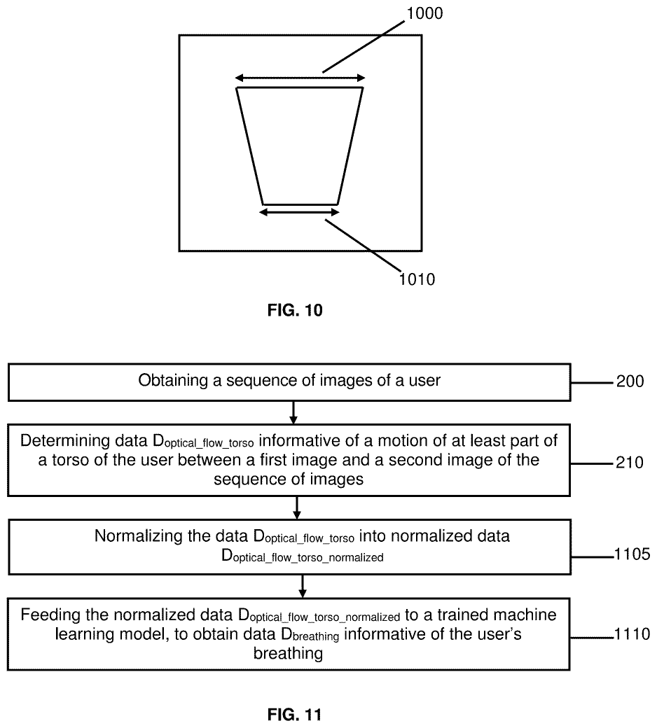

As visible in FIG. 10, the distance of the user from the acquisition device 100 can be estimated e.g., by determining the distance 1000 between the shoulders of the user in each image, and/or by determining the distance 1010 between the hips of the user in each image, and/or by using other relevant indicators.

When the distance of the user from the acquisition device 100 is too large in a given image, the given image can be disregarded (operation 930) in the following processing steps (computation of the optical flow and determination of the breathing data). Indeed, it can be expected that determination of the breathing data in the given image will not be sufficiently accurate.

The first threshold can be preset. It can be expressed e.g., as: an absolute value (maximal allowed distance between the user and the acquisition device 100), or as a minimal distance required between the shoulders of the user in the image (this reflects a maximal distance allowed between the user and the acquisition device 100), or as a minimal distance required between the hips of the user in the image (this also reflects a maximal distance allowed between the user and the acquisition device 100). Other types of thresholds can be defined.

As visible in FIG. 9, the data Dₚₒₛₑ can be used to determine (operation 940) whether the amplitude of the motion and/or of the speed of the user is above a certain threshold (second threshold) in one or more images of the sequence. Indeed, determination of the breathing data of the user in images in which the user is moving very quickly is expected to be less accurate. Therefore, it can be decided to disregard (operation 940) one or more images of the sequence in the following processing steps (computation of the optical flow and determination of the breathing data), based on this determination. Note that the various methods described herein are operative to determine the breathing data while the user is moving between the images. However, in some specific cases, in which there is much motion of the user, it can be decided to disregard the corresponding images.

The amplitude of the motion of the user can be determined based on the distance travelled by the body nodes of the user in the images. In some examples, the amplitude of the translation and/or of the rotation of the body nodes can be determined. The speed of the user can be determined by dividing the amplitude of the motion by the time between the images and the size of the user (to take into account for different distances of the user with respect to the acquisition device 100). In some examples, the speed of the user can be calculated based on the pose estimate of the user, the optical flow estimate, or a combination of both.

Attention is now drawn to FIG. 11, which describes an implementation of the method of FIG. 2, in which the optical flow field is normalized.

The method of FIG. 11 includes obtaining a sequence of images of a user (operation 200—already described with reference to FIG. 2) and determining (operation 210—already described with reference to FIG. 2) data D_{optical_flow_torso} informative of the motion of at least part of a torso of the user between a first image and a second image of the sequence of images. As mentioned above, data D_{optical_flow_torso} correspond to an optical flow field.

The method of FIG. 11 further includes normalizing the data D_{optical_flow_torso} into normalized data D_{optical_flow_torso_normalized}. This normalization can correspond to a spatial normalization (e.g., with respect to dimension(s) and/or orientation of the user) and/or to a temporal normalization (with respect to time, that it to say the time interval between the images for which the optical flow field is computed).

In some examples, the normalizing process enables reducing or cancelling the effect of one or more spatial parameters on the optical flow vectors (D_{optical_flow_torso}), such as size/scale of the user in the images, distance between the user and the acquisition device 100, orientation of the user in the images, etc. In some examples, the normalization is performed by normalizing a 2D optical flow field. As explained hereinafter, the 2D optical flow field can transformed/mapped into a 2D area.

In some examples, the time interval used to compute the optical flow vectors (D_{optical_flow_torso}) for different images of the sequence (with respect to other image(s) of the sequence) can be different among the different images: the normalizing process (with respect to time) enables reducing or cancelling the effect of this variation of the time interval among the different images.

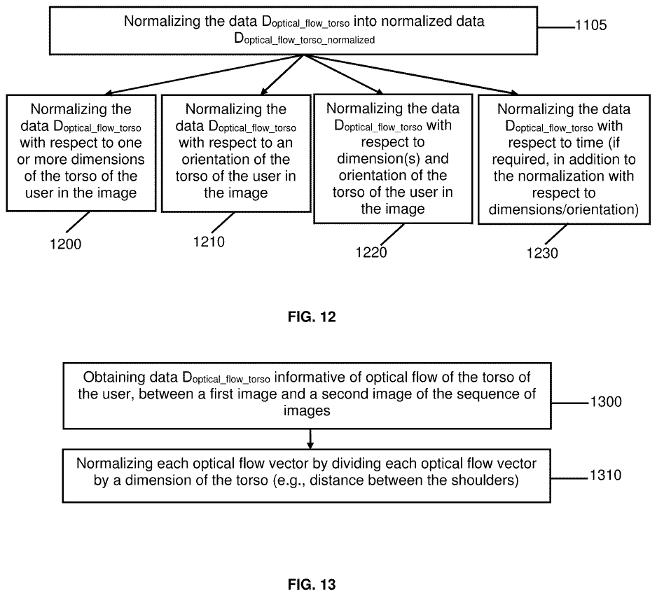

Various methods can be used to normalize the data D_{optical_flow_torso}, as visible in FIG. 12.

In some examples, normalizing the data D_{optical_flow_torso} comprises (operation 1200) normalizing the data D_{optical_flow_torso} with respect to one or more dimensions (or equivalent scale, or equivalent relative position of the user with respect to the acquisition device 100) of the user in the sequence of images.

The same motion of the torso of the user in the real world can appear differently in two different images of the sequence due to a different relative position of the user with respect to the camera (which induces the torso of the user to appear with different dimensions in the two different images). Variations can also exist between different users with different heights/body dimensions, although the same motion of the torso of the users has been performed in the real world.

As a consequence, for the same motion of the torso of the user due to breathing, different optical flow vectors are obtained in the data D_{optical_flow_torso}, due to the fact that the torso of the user appears with different dimensions in the two images. Normalization of the optical flow with respect to the dimensions of the torso and/or the scale of the torso in the image, enables obtaining the same (or nearly the same) optical flow vectors for the same motion of the torso of the user, irrespective of the distance between the user and the acquisition device 100. In other words, the data D_{optical_flow_torso_normalized} is invariant, or quasi-invariant, to the distance between the user and the acquisition device 100 (for the same motion of the torso of the user). Normalization with respect to the dimensions/scale of the user in the image is therefore achieved. Note that it has been mentioned above that when the user is so far from the acquisition device 100 that the image of the torso is not usable, the corresponding image can be disregarded. In this case, the optical flow is not determined and the normalization is not performed since the image of the user is not usable.

This normalization process can be performed by using the method of FIG. 13. Assume that data D_{optical_flow_torso} informative of optical flow of the torso of the user, between a first image and a second image of the sequence of images, is computed (operation 1300). A set of optical flow vectors is obtained. The method of FIG. 13 further includes (operation 1310) dividing each optical flow vector by a dimension of the torso in the first image (or in the second image). For example, all optical flow vectors are divided by the distance between the shoulders in the first image. As a consequence, the effect of the relative distance between the user and the acquisition device on the optical flow vectors is reduced or even cancelled (or other effects, due to the difference in height of the users). The normalized optical flow vectors are therefore insensitive/invariant, or quasi-insensitive/quasi-invariant, to the relative distance between the user and the acquisition device 100.

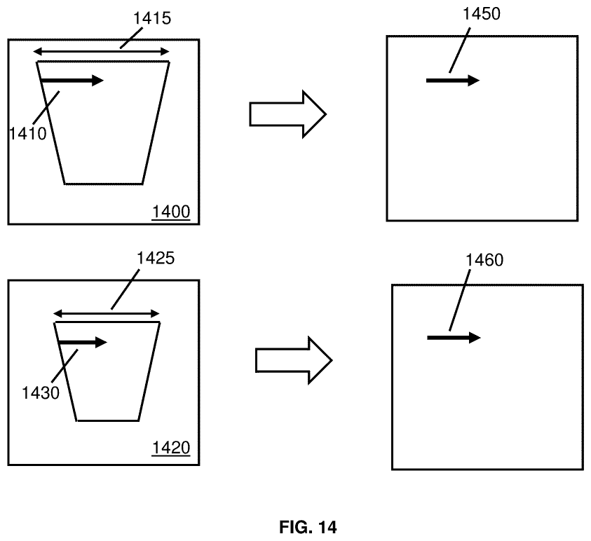

A non-limitative example of the method of FIG. 13 is provided in FIG. 14. Assume that an optical flow vector 1410 has been determined in image 1400. The optical flow vector 1410 is divided by the distance 1415 between the shoulders of the user in image 1400. The normalized optical flow vector 1450 is obtained. Assume that an optical flow vector 1430 has been determined in image 1420. The optical flow vector 1430 is divided by the distance 1425 between the shoulders of the user in image 1420. The normalized optical flow vector 1460 is obtained. By virtue of the normalization, a normalized optical flow vector with the same length is obtained. Indeed, the length of the normalized optical flow vector 1450 is the same as the length of the normalized optical flow vector 1460.

In some examples, normalizing the data D_{optical_flow_torso} comprises (operation 1210) normalizing the data D_{optical_flow_torso} with respect to an orientation of the torso of the user in the sequence of images. Note that in some examples, normalization is performed with respect to an orientation of the torso in the plane of the image (rotation of the torso around the normal to the image), but does not take into account rotations in other planes (e.g., anterior, posterior tilts of the torso).

The same motion of the torso of the user in the real world can appear differently in two different images of the sequence due to a different orientation of the torso of the user with respect to the camera (which induces the torso of the user to appear with different dimensions and/or a different shape in the two different images). As a consequence, for the same motion of the torso of the user due to breathing, different optical flow vectors are obtained in the data D_{optical_flow_torso}, due to the fact that the torso of the user appears with a different shape in the two images. Normalization of the optical flow with respect to the orientation of the torso in the image, enables obtaining the same (or nearly the same) optical flow vectors for the same motion of the torso of the user, irrespective of the orientation of the torso of the user with respect to the acquisition device 100. In other words, the data D_{optical_flow_torso_normalized} is invariant, or quasi-invariant, to the orientation of the torso of the user with respect to the acquisition device 100 (for the same motion of the torso of the user). In some examples, the data D_{optical_flow_torso_normalized} is invariant, or quasi-invariant, to a rotation of the torso with respect to a normal (normal vector) to the image.

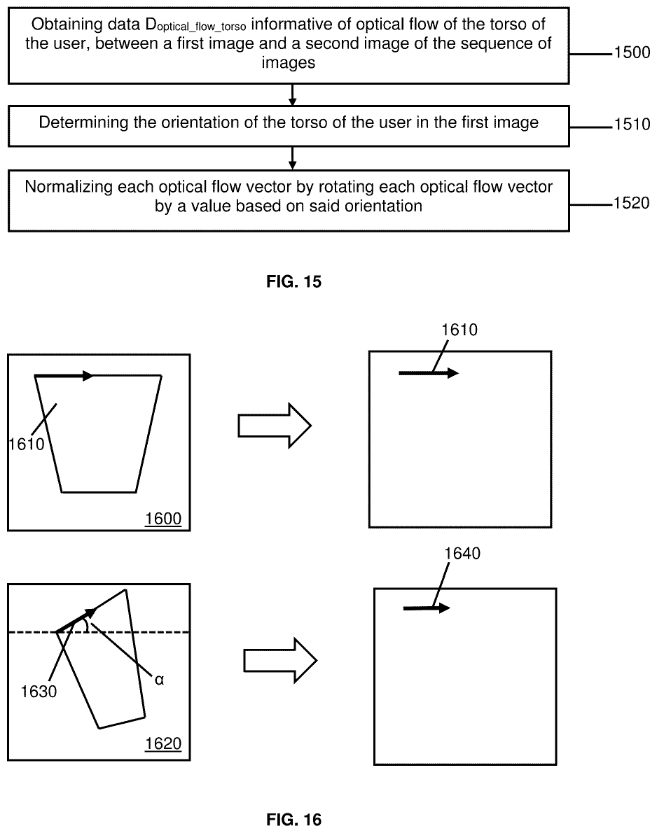

This normalization process can be performed by using the method of FIG. 15. Assume that data D_{optical_flow_torso} informative of optical flow of the torso of the user, between a first image and a second image of the sequence of images, is computed (operation 1500). A set of optical flow vectors is obtained. The method of FIG. 15 further includes (operation 1510) determining the orientation of the torso of the user in the first image. This orientation can be expressed e.g., as a deviation angle between the reference orientation, in which the user is standing still in front of the acquisition device 100, in a vertical plane. This orientation is an approximation expressed in a vertical plane (plane of the image). As explained with reference to FIG. 9, the position of the body nodes of the user can be determined in each image. This enables determining the position of the torso. In some examples, the orientation of the torso can be determined by computing the deviation of the line of the shoulders and/or of the line of the hips with respect to a horizontal line in the first image.

The method of FIG. 15 further includes normalizing (operation 1520) each optical flow vector by rotating each optical flow vector by a value determined based on said orientation. For example, assume that the orientation of the torso in the first image has been determined as equal to +α degrees with respect to the reference orientation of the torso. Then, the optical flow vectors can be rotated by an angle of −α degrees.

As a consequence, the effect of the orientation of the torso of the user (in the plane of the image) with respect to the acquisition device on the optical flow vectors is reduced, or even cancelled.

A non-limitative example of the method of FIG. 15 is provided in FIG. 16. Assume that an optical flow vector 1610 has been determined in the image 1600. Since orientation of the torso of the user in the image 1600 corresponds to the reference orientation, the optical flow vector 1610 does not need to be rotated in the normalized optical flow data.

Assume that an optical flow vector 1630 has been determined in image 1620. The optical flow vector 1630 is rotated to compensate for the inclination a of the line of the shoulders of the torso with respect to a horizontal axis. A normalized optical flow vector 1640 is obtained. By virtue of such normalization, a normalized optical flow vector 1640 with the same direction as the normalized optical flow vector 1610 is obtained.

In some examples, normalizing the data D_{optical_flow_torso} comprises (operation 1220) normalizing the data D_{optical_flow_torso} with respect to both the dimension(s) of the torso and orientation of the torso of the user in the sequence of images.

The same motion of the torso of the user in the real world can appear differently in two different images of the sequence due to a different relative position of the user with respect to the acquisition device 100, and due to a different orientation of the torso of the user with respect to the camera. This induces the torso of the user to appear with different dimensions and/or a different shape in the two different images. As a consequence, for the same motion of the torso of the user due to breathing, different optical flow vectors are obtained in the data D_{optical_flow_torso}, due to the fact that the torso of the user appears differently in the two images. Normalization of the optical flow with respect to the relative distance of the user and to the orientation of the torso (in the plane of the image) in the image, enables obtaining the same (or nearly the same) optical flow vectors for the same motion of the torso of the user, irrespective of the relative distance of the user and to the orientation of the torso of the user with respect to the acquisition device 100. In other words, the data D_{optical_flow_torso_normalized} is invariant, or quasi invariant, to the relative distance between the user and the acquisition device 100 and to the orientation of the torso of the user with respect to the acquisition device 100 (for the same motion of the torso of the user).

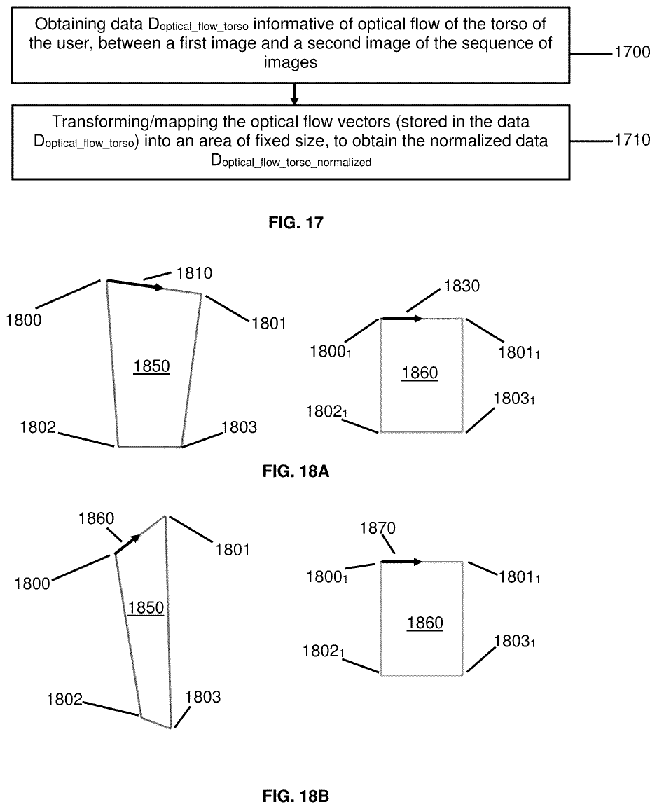

In some examples, this normalization process can be performed by using the method of FIG. 17.

Assume that data D_{optical_flow_torso} informative of optical flow of the torso of the user, between a first image and a second image of the sequence of images, is computed (operation 1700). A set of optical flow vectors is obtained. The method of FIG. 17 further includes (operation 1710) projecting the optical flow vectors (stored in the data D_{optical_flow_torso}) into an area of fixed size. For example, this area can correspond to a rectangle with fixed dimensions (fixed length and fixed width). The projection can be based on a transformation (also called homography) which enables converting predefined points of the torso of the user into predefined edges of the area of fixed size. For example, whatever the position and/or shape of the torso of the user, the two shoulders and the two hips of the user in the image are always converted into the respective edges of the area of fixed size. This projection enables obtaining D_{optical_flow_torso_normalized} which is invariant (or quasi-invariant) to the relative distance between the user and the acquisition device 100 and to the orientation of the torso of the user with respect to the acquisition device 100. This projection enables obtaining optical flow vectors which are insensitive to linear transformation of the torso of the user in the images (transformation in scale/size, and/or orientation).

Note that this projection is only an approximation, which assumes the torso of the user to be flat. In practice, the torso of the user is not perfectly flat, and therefore there can exist minor variations of the data D_{optical_flow_torso_normalized} depending on the relative distance between the user and the acquisition device 100 and/or on the orientation of the torso of the user with respect to the acquisition device 100.

A non-limitative example of the method of FIG. 17 is illustrated in FIGS. 18A and 18B. Assume that the projection mentioned in FIG. 17 converts the torso 1850 of the user as appearing in the image into an area of fixed size (rectangle 1860). In particular, the right shoulder 1800 of the user is converted into the left upper edge 18001 of the area 1860, the left shoulder 1801 of the user is converted into the right upper edge 18011 of the area 1860, the right hip 1802 of the user is converted into the left bottom edge 18021 of the area 1860, and the left hip 1803 of the user is converted into the right bottom edge 18031 of the area 1860. The optical flow vector 1810 (before normalization) is projected into the area 1860 of fixed size into the normalized flow vector 1830.

FIG. 18B depicts another image of the torso 1850 of the user. An optical flow vector 1860 has been computed. This optical flow vector 1860 corresponds, in the real world, to the same motion of the user's torso as the optical flow vector 1810. However, in practice, the optical flow vector 1860 has a different orientation and a different size in comparison to the optical flow vector 1810. This is due to the fact that the user has a different relative position and a different orientation with respect to the acquisition device 100 between the two images. The optical flow vector 1860 is projected into the area 1860 of fixed size and into the normalized optical flow vector 1870. As can be seen in FIGS. 18A and 18B, the same normalized optical flow vector is obtained, since the normalized optical flow vector 1860 and the normalized optical flow vector 1870 are identical.

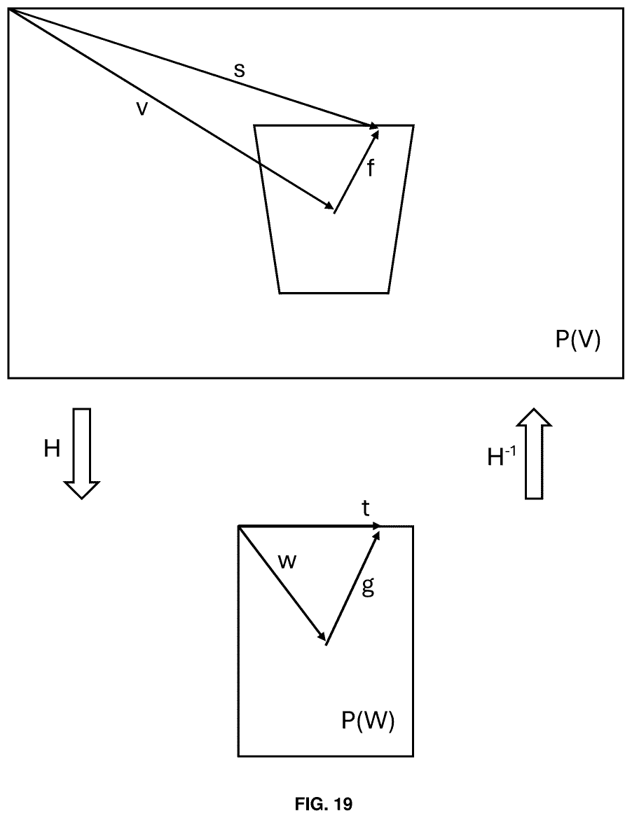

A possible implementation of the method of FIG. 17 will now be described with respect to FIG. 19.

Assume that it is desired to map/transform the vectors of the optical flow field (D_{optical_flow_torso}) from the image (the referential of the image is noted P(V)) into a rectangle of fixed size (the referential of the rectangle is noted P(W)). The edges (corners) of the rectangle have the following coordinates (matrix Q): Q1=(x′₁,y′₁), Q2=(x′₂,y′₂), Q3=(x′₃,y′₃) and Q4=(x′₄,y′₄). For example, Q1=(0,0), Q2=(0,5), Q3=(7,0), Q4=(7,5) (these values are not limitative).

Assume the torso is defined by four points P1 to P4 (matrix P), corresponding to the two shoulders and the two hips: P1=(x₁,y₁), P2=(x₂,y₂), P3=(x₃,y₃) and P4=(x₄,y₄).

A transformation matrix H (also called a homography) is determined, which enables converting P to Q. Computation of the transformation matrix is known per se. For example, in order to determine H, the following equation can be solved:

$$[ \begin{matrix}- {x}_{1} & - {y}_{1} & - 1 & 0 & 0 & 0 & - {x}_{1} {x}_{1}^{′} & {y}_{1} {x}_{1}^{′} & {x}_{1}^{′} \\ 0 & 0 & 0 & - {x}_{1} & - {y}_{1} & - 1 & {x}_{1} {y}_{1}^{′} & {y}_{1} {y}_{1}^{′} & {y}_{1}^{′} \\ - {x}_{2} & - {y}_{2} & - 1 & 0 & 0 & 0 & {x}_{2} {x}_{2}^{′} & {y}_{2} {x}_{2}^{′} & {x}_{2}^{′} \\ 0 & 0 & 0 & - {x}_{2} & - {y}_{2} & - 1 & {x}_{2} {y}_{2}^{′} & {y}_{2} {y}_{2}^{′} & {y}_{2}^{′} \\ - {x}_{3} & - {y}_{3} & - 1 & 0 & 0 & 0 & - {x}_{3} {x}_{3}^{′} & {y}_{3} {x}_{3}^{′} & {x}_{3}^{′} \\ 0 & 0 & 0 & - {x}_{3} & - {y}_{3} & - 1 & {x}_{3} {y}_{3}^{′} & {y}_{3} {y}_{3}^{′} & {y}_{3}^{′} \\ - {x}_{4} & - {y}_{4} & - 1 & 0 & 0 & 0 & - {x}_{4} {x}_{4}^{′} & {y}_{4} {x}_{4}^{′} & {x}_{4}^{′} \\ 0 & 0 & 0 & - {x}_{4} & - {y}_{4} & - 1 & {x}_{4} {y}_{4}^{′} & {y}_{4} {y}_{4}^{′} & {y}_{4}^{′}\end{matrix} ] [ \begin{matrix}{h}_{1} \\ {h}_{2} \\ {h}_{3} \\ {h}_{4} \\ {h}_{5} \\ {h}_{6} \\ {h}_{7} \\ {h}_{9}\end{matrix} ] = 0$$

$$H = [ \begin{matrix}{h}_{1} & {h}_{2} & {h}_{3} \\ {h}_{4} & {h}_{5} & {h}_{6} \\ {h}_{7} & {h}_{8} & {h}_{9}\end{matrix} ] , with \vert H \vert = 1$$

The inverse of the matrix H which enables converting the points from the area of fixed size to the image is noted H−1. The following operations can be then performed, for every point w in the area of fixed size:
Calculate its image location v, such that \hat{v}=H⁻¹*(wₓ, w_{y}, 1)^{T}, and

$$v = {( \frac{{\hat{v}}_{x}}{{\hat{v}}_{z}} , \frac{{\hat{v}}_{y}}{{\hat{v}}_{z}} )}^{T}$$

Determine the amplitude f of the optical flow at the location v in the image;
Determine the location s of the tip of the optical flow vector: s=v+f;
Project the location of the tip s back to the rectangle of fixed size: t=H*(s_{z}, s_{y}, 1)^{T}, and

$$t = {( \frac{{\hat{t}}_{x}}{{\hat{t}}_{z}} , \frac{{\hat{t}}_{y}}{{\hat{t}}_{z}} )}^{T}$$

Subtract n w to obtain the optical flow, normalized on the rectangle of fixed size: g=t−w. The normalized optical flow vector is therefore obtained (see vector \vec{g} in FIG. 19).

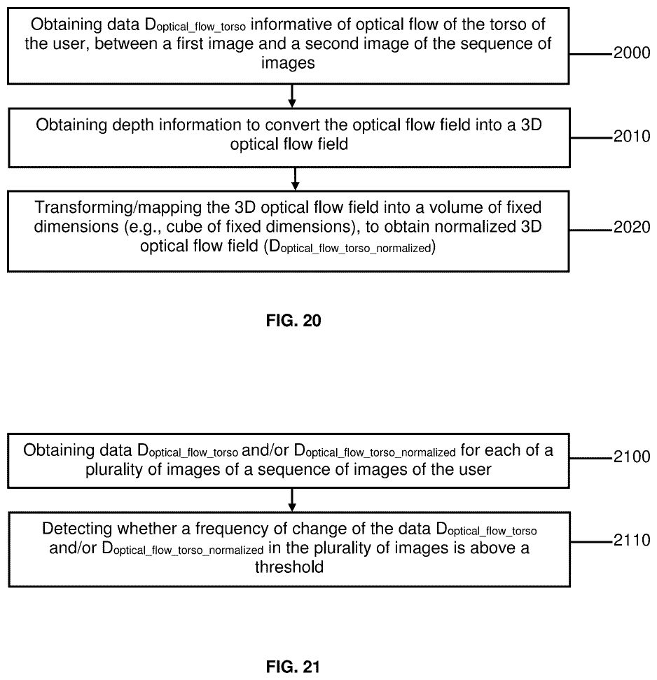

Attention is now drawn to FIG. 20, which describes a variant of the method of FIG. 17, performed in a 3D space.

Assume that data D_{optical_flow_torso} informative of the optical flow of the torso of the user, between a first image and a second image of the sequence of images, is computed (operation 2000). A set of optical flow vectors is obtained in a 2D space. It is possible to convert this set of optical flow vectors into a 3D space, by adding an additional parameter corresponding to the depth of each pixel (operation 2010). Various methods can be used to estimate depth from a 2D image (see e.g., “Real-Time Depth Estimation from 2D Images”, Jack Zhu and Ralph Ma). In some examples, a depth camera can be used to determine the depth of the pixels.

The method of FIG. 20 further includes transforming/mapping (operation 2020) the 3D optical vector into a volume of fixed dimensions, such as a cube of fixed dimensions.

This transformation/mapping can use a matrix, which converts any point in the camera referential to a person's reference coordinate system. The matrix can correspond to an affine transformation between the 3D location of the body nodes of the person, expressed in the camera referential, and points in a person's reference coordinate system. The person's reference coordinate system can be an arbitrary referential, such as a cube, in which it is defined where each body node, expressed in the camera referential, should be mapped. Determination of the matrix can use a least squares optimization. Note that the position of the body nodes, such as the hips and the shoulders, are not collocated with the surface of the torso. Indeed, these body nodes are located within the body. Since depth information is obtained, it is possible to convert the location of these body nodes on a surface of the torso. Without depth information an assumption can be made about the depth of the body joints within the body.

The following operations can be then performed, for every point w in the person's reference coordinate system, such as a cube of fixed dimensions (H is a four-by-four matrix):
Calculate its image location v in the camera referential, such that \hat{v}=H⁻¹*(w_{y}, w_{y}, w_{z}, 1)^{T}, and v=(\hat{v}ₓ, \hat{v}_{y}, \hat{v}_{z})^{T}
Determine the amplitude f of the optical flow at the location v;
Determine the location s of the tip of the optical flow vector: s=v+f;
Project the location of the tip s back to the rectangle of fixed size: \hat{t}=H*(sₓ, s_{y}, s_{z}, 1)^{T}, and t=(\hat{t}ₓ, \hat{t}_{y}, \hat{t}_{z})^{T}
Subtract w to obtain the optical flow, normalized on the cube of fixed dimensions: g=t−w. The normalized 3D optical flow vector g is therefore obtained.

The normalized 3D optical flow vectors can be then fed to the machine learning model 150, in order to predict data informative of the user's breathing, as explained with reference to FIG. 2.

As visible in FIG. 12, in some examples, normalizing the data D_{optical_flow_torso} comprises (operation 1230) normalizing the data D_{optical_flow_torso} with respect to time. As mentioned above, the data D_{optical_flow_torso} includes, for each given image of the sequence, one or more optical flow fields between the given image and one or more other image(s) of the sequence. A plurality of optical fields is therefore computed, and fed to the machine learning model 150. Each optical flow field is determined between two images separated by a certain time interval (corresponding to the difference in the time of acquisition of the two images). It can occur that this time interval is not constant among the different optical fields. This can be due to various reasons, such as variable frame rates, loss of images, usage of variable time intervals, etc. The temporal normalization of an optical flow field, determined between two images separated by a certain time interval ΔT, can be based on a ratio between the optical flow field and the time interval ΔT. In particular, the optical flow field can be divided by the value ΔT. For each optical flow field (associated with a certain time interval ΔT), the amplitude of each optical vector of the optical flow field is divided by the value ΔT. This enables normalization of the optical flow field with respect to time. Note that this is possible to normalize the optical flow field(s) both with respect to time and with respect to one or more spatial parameters (dimensions of the torso of the user and/or orientation of the torso of the user). In this case, both the spatial normalization (as described above) and the temporal normalization (as described above) are performed. It is also possible to perform a spatial normalization (without performing a temporal normalization), or temporal normalization (without performing spatial normalization).

Attention is now drawn to FIG. 21. Assume that the data D_{optical_flow_torso} and/or D_{optical_flow_torso_normalized} have been computed for each of a plurality of images of the sequence (operation 2100). In other words, the optical flow (or normalized optical flow) has been computed for different images of the sequence.

The method of FIG. 21 further includes (operation 2110) detecting whether a frequency of change of the data D_{optical_flow_torso} and/or D_{optical_flow_torso_normalized} in the plurality of images is above a threshold. The threshold can be preset. A high frequency of change is indicative that the environment in which the user is evolving is dark, which induces noise in the optical flow vectors, and, in turn, a high frequency of change in the optical flow vectors. In response to this detection, it can be decided not to feed the data D_{optical_flow_torso} and/or D_{optical_flow_torso_normalized} for which the frequency of change is above the threshold. Indeed, it can be expected that the accuracy of the determination of the breathing data will not be satisfactory, due to the darkness of these images.

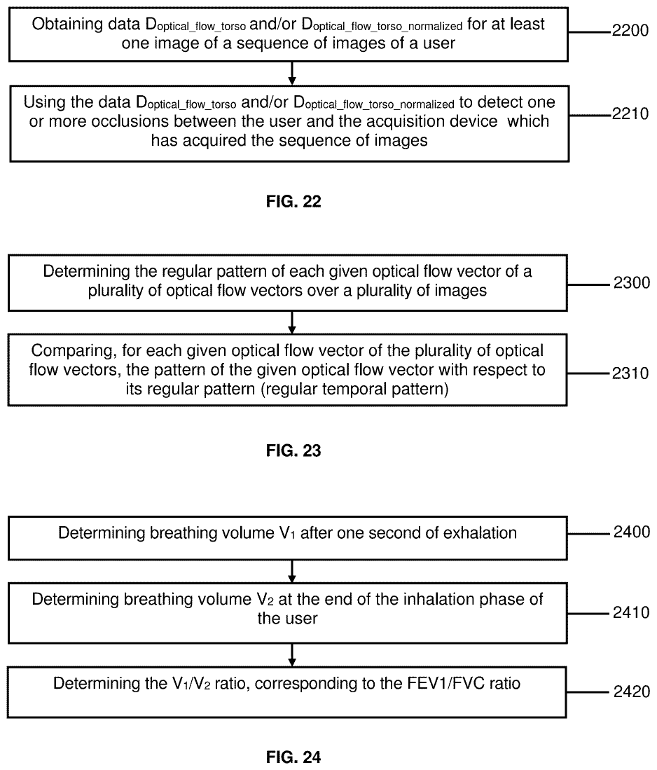

Attention is now drawn to FIG. 22. Assume that the data D_{optical_flow_torso} and/or D_{optical_flow_torso_normalized} have been computed for at least one image of the sequence of images (operation 2200), with respect to another image of the sequence. In other words, the optical flow (or normalized optical flow) has been computed for at least one image (with respect to another image of the sequence).

The method of FIG. 22 further includes using (operation 2210) the data D_{optical_flow_torso} and/or D_{optical_flow_torso_normalized} to detect one or more occlusions between the user and the acquisition device 100 which has acquired the sequence of images. This detection can include detecting whether one or more occlusions are present, and their location in the image. In particular, operation 2210 can include detecting that a set of optical flow vectors has an amplitude equal to zero, or is below a certain threshold. This indicates that an occlusion is present, such as a wall, etc. The occlusion corresponds to a static occlusion (a fixed occlusion, such as wall), or to an occlusion which is temporarily static in a few images. If this occlusion is present at a relevant location, such as the torso of the user, it can be decided not to feed the data D_{optical_flow_torso} and/or D_{optical_flow_torso_normalized} of the corresponding image(s). Indeed, it can be expected that the accuracy of determination of the breathing data will not be satisfactory, due to the presence of the occlusion in these images.

Attention is now drawn to FIG. 23.

The method of FIG. 23 includes determining (operation 2300) the pattern of each given optical flow vector of a plurality of optical flow vectors over a plurality of images, and comparing (operation 2310), for each given optical flow vector of the plurality of optical flow vectors, the pattern of the given optical flow vector with respect to its regular pattern (regular temporal pattern).

The pattern of a given optical flow vector (with a tail at given location) corresponds to the orientation of the given optical flow vectors (with a tail at this location) over time. The regular temporal pattern of an optical flow vector with a tail at a given location can be computed by extracting the optical flow vectors over a plurality of images, with a tail at this given location.

If the pattern of the given optical flow vector differs from its regular temporal pattern, this can indicate that this given optical flow vector is noisy. This can be detected by using an outlier detector, such as an Autoencoder. This is not limitative and other algorithms can be used. It can be decided to set the noisy optical flow vector(s) to zero, in order to avoid feeding noisy data to the machine learning model 150.

Attention is now drawn to FIG. 24.

In some examples, the machine learning model 150 is configured to output the (as part of the data D_{breathing}) inhalation phases and/or exhalation phases of the user. In particular, it can output the beginning and/or the end of the inhalation phase, and/or the beginning and/or the end of the exhalation phase of the user.

An interesting ratio in the medical field is the FEV1/FVC ratio (also called modified Tiffeneau-Pinelli index). FEV1 is the breathing volume after is of exhalation. FEV is the breathing volume after a full inhalation.

When the beginning of the exhalation phase of the user has been detected by the machine learning model 150, the breathing volume V₁ after one second of exhalation (as output by the machine learning model 150) can be output by the machine learning model 150. In some examples, V₁ is a relative value and therefore corresponds to FEV1 multiplied by a certain unknown factor K (operation 2400).

When the end of the inhalation phase of the user has been detected by the machine learning model 150, the breathing volume V₂ (as output by the machine learning model 150) can be obtained. In some examples, V₂ is a relative value and therefore corresponds to FVC multiplied by the same unknown factor K (operation 2410).

The ratio V₁/V₂ corresponds to the FEV1/FVC ratio (operation 2420). As can be seen, the unknown factor K has been cancelled by the division of the two values.

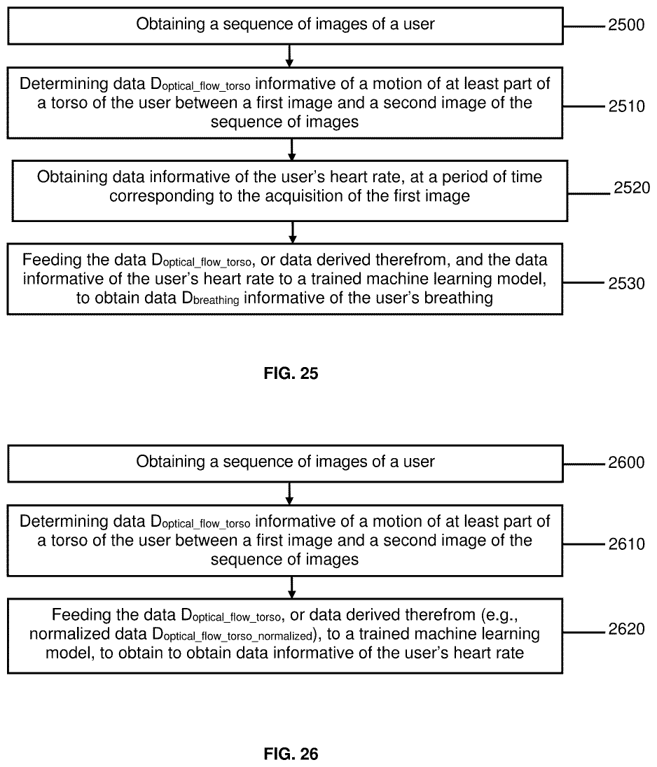

Attention is now drawn to FIG. 25.

The method of FIG. 25 includes obtaining (operation 2500) a sequence of images of a user and determining data D_{optical_flow_torso} informative of a motion of at least part of a torso of the user between a first image and a second image of the sequence of images (operation 2510). As mentioned above, data D_{optical_flow_torso} can include determining optical flow vectors. The method of FIG. 25 includes obtaining (operation 2520) data informative of the user's heart rate. In some examples, the data informative of the user's heart rate can correspond to a period of time at which the first image has been acquired. Acquisition of data informative of the user's heart rate can be performed using various methods, such as Pulse oximeters, Electrocardiograph machines, Ballistocardiography methods, Remote Photoplethysmography (rPPG), etc. In some examples, data informative of the user's heart rate (which is fed to the machine learning model 150 as explained hereinafter) is obtained in the frequency domain and/or as a time series. In some examples, data informative of the user's heart rate corresponds to raw data (as measured using one or more of the methods above), or to filtered data.

The method of FIG. 25 further includes feeding (operation 2530) the data D_{optical_flow_torso}, or data derived therefrom (normalized data D_{optical_flow_torso_normalized}), and the data informative of the user's heart rate to the trained machine learning model 150, to obtain data D_{breathing} informative of the user's breathing. Indeed, the heart rate is correlated to the breathing of the user, and therefore, it can be expected that this additional input (the user's heart rate) will improve the ability of the machine learning model 150 to determine the breathing data of the user. Note that in some examples, the machine learning model 150 has been trained to predict data informative of the user's breathing with training data including optical flow data and heart rate data. Therefore, once the machine learning model 150 has been trained, it is able to receive both optical flow data and heart rate data and to predict user's breathing data.

Attention is now drawn to FIG. 26, which describes a method of determining data informative of a user's heart rate.

The method of FIG. 26 includes obtaining (operation 2600) a sequence of images (also called frames) of a user. The sequence of images has been acquired e.g., by the acquisition device 100. Operation 2600 is similar to operation 200 and is therefore not described again (it is possible to refer to the description of operation 200 provided above). Note that the sequence of images can be pre-processed, as explained above.

The method of FIG. 26 further includes determining (operation 2610) data D_{optical_flow_torso} informative of the motion of at least part of a torso of the user between the first image and the second image. In particular, operation 2610 can include determining the optical flow between at least part of the first image and at least part of the second image. Operation 2610 is similar to operation 210 and is therefore not described again (it is possible to refer to the description of operation 210 provided above).

In some examples, the data D_{optical_flow_torso} can be normalized into normalized data D_{optical_flow_torso_normalized}, as explained with reference to FIGS. 12 to 20. The same methods can be used herein and are not described again. Normalization can be performed such that the optical flow is normalized with respect to dimension(s) of the torso of the user (or in a similar way, to the relative distance between the user and the acquisition device 100) and/or the orientation of the torso of the user.

The method of FIG. 26 further includes feeding (operation 2620) the data D_{optical_flow_torso} to the machine learning model 150. In some examples, the method of FIG. 26 includes feeding data derived from the data D_{optical_flow_torso} to the machine learning model 150. In particular, the normalized data D_{optical_flow_torso_normalized}, which has been derived from the data D_{optical_flow_torso}, can be fed to the machine learning model 150. In some examples, both the data D_{optical_flow_torso} and the data D_{optical_flow_torso_normalized} can be fed to the machine learning model 150.

This enables obtaining data informative of the user's heart rate. In some examples, the output of the machine learning model 150 corresponds to the user's heart rate. Indeed, the machine learning model 150 may have been trained to detect heartbeats (corresponding to certain displacements of the human's torso) and this allows it to deduce the user's heart rate, or other metrics informative of the user's heart rate (such as heart rate variability—HRV, or any other related metrics informative of the user's heart rate) by computing the time between consecutive heart beats. Heart rate variability (HRV) is the physiological phenomenon of variation in the time interval between heartbeats. It is measured by the variation in the beat-to-beat interval. In some examples, for each image of the sequence for which the optical flow has been determined and fed to the machine learning model 150, the machine learning model 150 outputs the user's estimated heart rate, or any other related metrics.

Note that the method of FIG. 26 can be performed in real time or quasi-real time, while the sequence of images is acquired by the acquisition device 100. In addition, the method of FIG. 26 can be performed repeatedly for different images of the sequence. For each given image of the sequence, the corresponding optical flow is computed with respect to another image acquired after the given image. The corresponding optical flow field, or data derived therefrom (normalized optical flow) is then fed to the machine learning model 150 to generate data informative of the user's heart rate. In other words, for each image of the sequence acquired at a given time t, the corresponding heart rate of the user at time t is estimated. Note that the user's heart rate as output by the machine learning model 150 can correspond to an absolute number.

In some examples, data informative of the user's heart rate can be output, such as on the output device 160. Display of the user's heart rate can be performed using different methods, such as display of a value on the screen, display of one or more indicators indicative of the user's heart rate, display of a relative value using a scale between a minimal value and a maximal value, etc. In some examples, display of the user's heart rate includes displaying an animation, which differs depending on the user's heart rate. For example, the higher the user's heart rate, the higher the amplitude and/or velocity and/or changing frequency of the motion of the animation. In some examples, an alert can be raised when the user's heart rate is above a threshold. Data informative of the user's heart rate can be output using an audio feedback.

In some examples, for each image for which the data informative of the user's heart rate has been computed, data informative of the user's heart rate can be displayed together with the image. Data informative of the user's heart rate can be superimposed on the image. In some examples, the data D_{optical_flow_torso} or the data D_{optical_flow_torso_normalized} can be also displayed, e.g., on the image for which the data D_{optical_flow_torso} or D_{optical_flow_torso_normalized} has been computed.

In some examples, data informative of the heart rate (or the user's breathing) can be measured using two methods:
a first method uses Remote Photoplethysmography (rPPG), or any other relevant method, in order to output data informative of the heart rate (or data informative of the user's breathing). This method enables measuring cardiac activity based on images of the face of the user (this method uses a signal informative of blood flow generated based on the change in the colour on the human's skin). The cardiac activity can be converted into data informative of the user's breathing, since the heart beats (in particular, their intervales and/or amplitudes) are correlated to the respiration phases of the user;
a second method uses the various solutions described herein, in which the machine learning model 150 outputs data informative of the heart rate (or data informative of the user's breathing) based on images of the torso of the user.

This combination provides several technical advantages. The rPPG (or other existing methods) is not always capable of outputting the data informative of the user's heart rate (and/or data informative of the user's breathing) in real time. The proposed solution compensates for this drawback. On the other hand, the proposed solution can use the data measured by the rPPG solution (or provided by other existing methods enabling measurement of data informative of the user's heart rate, and/or data informative of the user's breathing).

In some examples, data informative of the heart rate (or data derived therefrom, such as data informative of the user's breathing), as measured using rPPG (or using another method, such as a remote measurement method) can be fed to the machine learning model 150, in order to generate another estimate (refined estimate) of the data informative of the user's heart rate (and/or of the data informative of the user's breathing).

In some examples, another machine learning model (e.g., neural network, deep learning neural network, temporal model, or any other adapted model) receives data informative of the user's heart rate (and/or data derived therefrom, such as data informative of the user's breathing) measured using rPPG (or using another method, such as a remote measurement method) and data informative of the user's heart rate (and/or data informative of the user's breathing) output by the machine learning model 150, and generates an updated estimate of the data informative of the user's heart rate (and/or an updated estimate of the data informative of the user's breathing).

Note that it is possible to use at least two different machine learning models which operate in parallel: a first machine learning model is used to determine data informative of the user's breathing (using the various methods described herein) and a second machine learning model is used to determine data informative of the user's heart rate (as explained above). Both output (breathing volume, or other breathing data, together with the user's heart rate) can be displayed on the image. In some other examples, the same machine learning model is used to determine both the breathing data and the user's heart rate.

As can be understood from the description above, the various method(s) and/or system(s) enable obtaining the data D_{breathing} informative of the user's breathing (and the data informative of the user's heart rate) without requiring the user to take part to a calibration process. In particular, the user is not required to stand, lie or sit still in front of the acquisition device and/or to perform specific predefined calibration activities. Note that, as explained hereinafter, the determination of the absolute breathing volume of the user may require the user to provide data informative of his breathing volume, and/or to undergo measurements of his breathing volume. This is however not mandatory.

Similarly, according to some examples, the various method(s) and/or system(s) enable obtaining the data D_{breathing} informative of the user's breathing (and the data informative of the user's heart rate) in at least one given image (or more), even if the user was absent from images preceding the given image in the sequence, without requiring the user to take part to a calibration process. In other words, even if the user has moved out from the field of view of the acquisition device within certain frames, there is no need to perform afterwards a calibration with respect to the user in order to estimate the biological data of the user in subsequent frames (acquired after the certain frames), in which the user is present. In other words, the solution enables self-calibration which adapts automatically to the user.

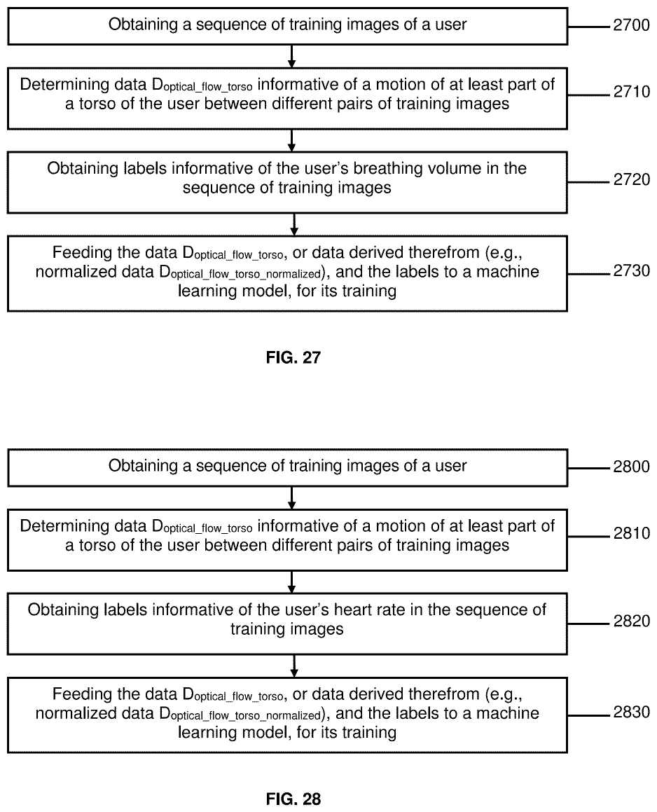

Attention is now drawn to FIG. 27, which describes a method of training the machine learning model 150.

The method includes obtaining (operation 2700) a sequence of training images of one or more users, acquired by an acquisition device (such as the acquisition device 100, or another acquisition device). Note that it is possible to obtain a plurality of different sequences of training images, each informative of a different user.

The sequence(s) of training images is/are processed. Assume that the sequence of training images includes images I₁ to I_{N}. For each training image Iⱼ of the sequence, data D_{optical_flow_torso_Ij} informative of the motion of at least part of a torso of the user between the training image Iⱼ and another training image Iₖ (acquired after the training image Iₖ, with k>j), is computed (operation 2710). As explained above, the data D_{optical_flow_torso_Ij} can correspond to the optical flow between the training image Iⱼ and the other training image Iₖ.

In some examples, for each training image Iⱼ, the data D_{optical_flow_torso_Ij} can be normalized, in order to obtain normalized data D_{optical_flow_torso_normalized_Ij}. Various methods have been provided above (see e.g., FIGS. 12 to 20) and can be used herein.

For each training image Iⱼ, a label can be determined. This label can be provided e.g., by an operator and/or by using one or more sensors enabling measuring data informative of the user's breathing. A set of labels is obtained for the sequence of training images (operation 2720).

In some examples, the operator can indicate the breathing volume of the user in the training image Iⱼ, which is then used as a label. This breathing volume can correspond to a relative breathing volume. Different definitions can be used to label the breathing volume in the training images. In some examples, the breathing volume is informative of the volume of air that has been inhaled by the user (how much air is present in the lungs): it is maximal at the end of a full inhalation, and minimal at the end of a full exhalation.

In some examples, the breathing volume is informative of the volume of air that has been exhaled by the user (i.e., how much air is outside of the lungs): it is maximal at the end of a full exhalation, and minimal at the end of a full inhalation.

Other definitions of the breathing volume can be used, depending on the needs.

The machine learning model 150 is fed with the set of data D_{optical_flow_torso_Ij} determined for the sequence of training images, and/or with the set of normalized data D_{optical_flow_torso_normalized_Ij} determined for the sequence of training images, together with the set of labels. The machine learning model 150 is trained (using a loss function) to determine the breathing volume in the sequence of training images, based on the input received during its training (operation 2730).

In some examples, the sequence of training images is divided into chunks of images. Each chunk has a certain duration (for example, 10s—this value being not limitative). For each chunk of images, a corresponding optical flow field is determined with respect to another image of the chunk. An operator can annotate (label) the optical flow field corresponding to the maximal exhalation and the optical flow field corresponding to the minimal exhalation. Note that the maximal inhalation is not necessarily a full inhalation and that the maximal exhalation is not necessarily a full exhalation. Indeed, the maximal exhalation and the maximal inhalation are determined as relative values, within each respective chunk of images. This training enables the machine learning model to operate in the first mode mentioned above, in which it adapts to the breathing mode of the user over time.

In some examples, the machine learning model 150 is trained without necessarily dividing the sequence of images into chunks. In this case, for a certain sequence of training images, the maximal inhalation and the maximal exhalation are identified over the sequence and labelled (see second mode above).

In some examples, the machine learning model 150 can be trained to output a value which is a percentage of the absolute breathing volume (see the second and third modes mentioned above). For example, during the training phase, the user is asked to perform at least once a full inhalation and a full exhalation. The training images (or corresponding optical flow fields) are then labelled and fed to the machine learning model 150 for its training. In addition, for each other training image (or corresponding optical flow fields), a label can be generated, which is informative of the breathing volume of the user in this training image, expressed as a percentage of the absolute breathing volume of the user. This label can be obtained e.g., by using a spirometer connected to the user during the training phase.

In some examples, the machine learning model 150 is fed, for each training image, with two optical flows. A first optical flow field corresponds to the optical flow field between the current training image and the training image of a user corresponding to a full inhalation (in some examples, this image can be identified by asking the user to provide an indication to the system when he has reached a full inhalation). A second optical flow field corresponds to the optical flow field between the current training image and the image of the user corresponding to a full exhalation (in some examples, this image can be identified by asking the user to provide an indication to the system when he has reached a full exhalation). In addition, for each training image, a label is generated, which is informative of the breathing volume of the user in this training image, expressed as a percentage of the absolute breathing volume of the user. This label can be obtained e.g., by using a spirometer connected to the user during the training phase. Note that as mentioned above, the user(s) which is/are present in the training images can be different from the users present in the prediction phase. After this training phase, the trained machine learning model 150 is operative to provide, for images of a user, the breathing volume expressed relative to the maximal breathing volume of the user. As mentioned above, if a specific user has provided his maximal breathing volume, this output can be then converted into an actual breathing volume (in Liters, or other relevant units).

As mentioned above, it is possible to train the machine learning model 150 with additional data, such as the user's heart rate (or other metrics, such as HRV). This additional data is fed to the machine learning model 150 in addition to the data mentioned above. This data can be measured by using a medical device (see examples above). This data can be measured while the sequence of images of the user is acquired by the acquisition device 100.

In some examples, it is possible to train different machine learning models, for predicting data informative of the user's breathing, and/or data informative of the user's rate, which can operate in parallel and/or in cascade.

Attention is now drawn to FIG. 28, which describes another method of training the machine learning model 150.

The method includes obtaining (operation 2800) a sequence of training images of one or more users, acquired by an acquisition device (such as the acquisition device 100, or another acquisition device).

The sequence of training images is processed. Assume that the sequence of training images includes images I₁ to I_{N}. For each training image Iⱼ of the sequence, data D_{optical_flow_torso_Ij} informative of a motion of at least part of a torso of the user between the training image Iⱼ and another training image Iₖ (acquired after the training image Iₖ, with k>j) is computed (operation 2810). As explained above, the data D_{optical_flow_torso_Ij} can correspond to the optical flow between the training image Iⱼ and the other training image Iₖ.

In some examples, for each training image Iⱼ, the data D_{optical_flow_torso_Ij} can be normalized, in order to obtain normalized data D_{optical_flow_torso_normalized_Ij}. Various methods have been provided above (see e.g., FIGS. 12 to 20) and can be used herein.

For each training image Iⱼ, a label can be determined. The label indicates whether a heart beat of the user has occurred in the image, or not. This label can be provided e.g., by an operator and/or by using one or more sensors enabling measuring the user's heart beat/heart rate (see examples of device above). A set of labels is obtained for the sequence of training images (operation 2820).

The machine learning model 150 is fed with the set of data D_{optical_flow_torso_Ij} determined for the sequence of training images, and/or with the set of normalized data D_{optical_flow_torso_normalized_Ij} determined for the sequence of training images, together with the set of labels. The machine learning model 150 is trained (using a loss function) to determine the user's heart rate (corresponding to the time between two consecutive heart beats) in the sequence of training images, based on the input received during its training (operation 2830).

Note that it is possible to train the machine learning model 150 to both predict data informative of the user's breathing and data informative of the user's heart rate. It is also possible to train two different machine learning models: a first machine learning model is trained to predict data informative of the user's breathing and a second machine learning model is trained to predict data informative of the user's heart rate.

Unless specifically stated otherwise, as apparent from the following discussions, it is appreciated that throughout the specification discussions utilizing terms such as, “obtaining”, “determining”, “feeding”, “training”, “outputting”, “normalizing”, “generating”, “estimating”, “using”, “detecting”, or the like, refer to the action(s) and/or process(es) of a computer (and/or one or more processing circuitries) that manipulate and/or transform data into other data, said data represented as physical, such as electronic, quantities and/or said data representing the physical objects.

The computer and/or the data processing circuitry (designated also as processing circuitry) can comprise, for example, one or more processors operatively connected to computer memory, loaded with executable instructions for executing operations, as further described below. The data processing circuitry encompasses a single processor or multiple processors, which may be located in the same geographical zone, or may, at least partially, be located in different zones, and may be able to communicate together. The one or more processors can represent one or more general-purpose processing devices such as a microprocessor, a central processing unit, or the like. More particularly, a given processor may be one of: a complex instruction set computing (CISC) microprocessor, a reduced instruction set computing (RISC) microprocessor, a very long instruction word (VLIW) microprocessor, a processor implementing other instruction sets, or a processor implementing a combination of instruction sets. The one or more processors may also be one or more special-purpose processing devices such as an application specific integrated circuit (ASIC), a field programmable gate array (FPGA), a digital signal processor (DSP), a network processor, or the like. The one or more processors are configured to execute instructions for performing the operations and steps discussed herein.

In the detailed description, numerous specific details have been set forth in order to provide a thorough understanding of the disclosure. However, it will be understood by those skilled in the art that the presently disclosed subject matter may be practiced without these specific details. In other instances, well-known methods, procedures, components, and circuits have not been described in detail so as not to obscure the presently disclosed subject matter.

The memories referred to herein can comprise one or more of the following: internal memory, such as, e.g., processor registers and cache, etc., main memory such as, e.g., read-only memory (ROM), flash memory, dynamic random access memory (DRAM) such as synchronous DRAM (SDRAM) or Rambus DRAM (RDRAM), etc.

The terms “non-transitory memory” and “non-transitory computer readable medium” used herein should be expansively construed to cover any volatile or non-volatile computer memory suitable to the presently disclosed subject matter. The terms should be taken to include a single medium or multiple media (e.g., a centralized or distributed database, and/or associated caches and servers) that store the one or more sets of instructions. The terms shall also be taken to include any medium that is capable of storing or encoding a set of instructions for execution by the computer and that cause the computer to perform any one or more of the methodologies of the present disclosure. The terms shall accordingly be taken to include, but not be limited to, a read only memory (“ROM”), random access memory (“RAM”), magnetic disk storage media, optical storage media, flash memory devices, etc.

It is to be noted that while the present disclosure refers to the processing circuitry 120 being configured to perform various functionalities and/or operations, the functionalities/operations can be performed by the one or more processors of the processing circuitry 120 in various ways. By way of example, the operations described hereinafter can be performed by a specific processor, or by a combination of processors. The operations that have been described can thus be performed by respective processors (or processor combinations) in the processing circuitry 120, while, optionally, at least some of these operations may be performed by the same processor. The present disclosure should not be limited to be construed as one single processor always performing all the operations.

It is appreciated that, unless specifically stated otherwise, certain features of the presently disclosed subject matter, which are described in the context of separate embodiments, can also be provided in combination in a single embodiment. Conversely, various features of the presently disclosed subject matter, which are described in the context of a single embodiment, can also be provided separately, or in any suitable sub-combination. In the detailed description, numerous specific details are set forth in order to provide a thorough understanding of the methods and apparatus.

In embodiments of the presently disclosed subject matter, fewer, more, and/or different stages than those shown in the methods of FIGS. 2, 9, 11, 12, 13, 15, 17, 20 to 28 may be executed. In embodiments of the presently disclosed subject matter, one or more stages illustrated in the methods of FIGS. 2, 9, 11, 12, 13, 15, 17, 20 to 28 may be executed in a different order, and/or one or more groups of stages may be executed simultaneously.

It is to be understood that the invention is not limited in its application to the details set forth in the description contained herein or illustrated in the drawings.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

Those skilled in the art will readily appreciate that various modifications and changes can be applied to the embodiments of the invention as hereinbefore described without departing from its scope, defined in and by the appended claims.

What is claimed is:

1. A system comprising one or more processing circuitries configured to:
obtain a sequence of images of a user, the sequence of images comprising a first image and a second image,
determine data D_{optical_flow_torso} informative of a motion of at least part of a torso of the user between the first image and the second image, and
feed the data D_{optical_flow_torso}, or data derived from the data D_{optical_flow_torso}, to at least one machine learning model, to obtain data D_{breathing} informative of the user's breathing.

2. The system of claim 1, wherein determining data D_{optical_flow_torso} comprises at least one of (i) or (i):
(i) determining an optical flow field between at least part of the first image and at least part of the second image;
(ii) determining a first optical flow field between at least part of the first image and at least part of the second image, and determining a second optical flow field between at least part of the first image and at least part of another image of the sequence, different from the first and second images.

3. The system of claim 1, wherein the at least one machine learning model comprises a temporal machine learning model.

4. The system of claim 1, wherein at least one of (i), (ii), (iii) or (iv) is met:
(i) the data D_{breathing} is informative of breathing volume of the user;
(ii) the data D_{breathing} is informative of breathing strength of the user;
(iii) the data D_{breathing} is informative of breathing rate of the user;
(iv) the data D_{breathing} is informative of breathing phase of the user.

5. The system of claim 1, configured to:
normalize the data D_{optical_flow_torso} into normalized data D_{optical_flow_torso_normalized}, and
feed the data D_{optical_flow_torso_normalized} to the at least one machine learning model, to obtain the data D_{breathing} informative of the user's breathing.

6. The system of claim 5, wherein normalizing the data D_{optical_flow_torso} into the data D_{optical_flow_torso_normalized} comprises performing at least one of:
(i) a normalization of the data D_{optical_flow_torso} with respect to at least one of: one or more dimensions of the user, or an orientation of the user; or
(ii) a normalization of the data D_{optical_flow_torso} with respect to time.

7. The system of claim 5, wherein normalizing the data D_{optical_flow_torso} into the data D_{optical_flow_torso_normalized} comprises normalizing the data D_{optical_flow_torso} with respect to at least one of:
one or more dimensions of the user in one or more images of the sequence of images, or
an orientation of the user in one or more images of the sequence of images.

8. The system of claim 5, wherein the data D_{optical_flow_torso} comprises an optical flow field between the first image acquired at time t₁, and the second image acquired at time t₂, wherein said normalizing of the data D_{optical_flow_torso} into the data D_{optical_flow_torso_normalized} is based on a ratio between the optical flow field and a time interval between t₁ and t₂.

9. The system of claim 1, configured to:
generate data D_{optical_flow_torso_normalized} based on a transformation of the data D_{optical_flow_torso} into an area of fixed size, and
feed the data D_{optical_flow_torso_normalized} to the at least one machine learning model, to obtain the data D_{breathing} informative of the user's breathing.

10. The system of claim 9, wherein said transformation enables converting predefined points of the torso of the user into predefined edges of the area of fixed size.

11. The system of claim 1, configured to use at least part of the sequence of images acquired by an acquisition device to determine data Dₚₒₛₑ informative of body nodes of the user, and use the data Dₚₒₛₑ to perform at least one of (i) or (ii):
(i) detecting whether the distance of the user from the acquisition device is above a first threshold;
(ii) detecting whether an amplitude or a speed of a motion of the user is above a second threshold.

12. The system of claim 1, wherein the first image and the second image have been acquired by an acquisition device, wherein the first image and the second image have not been acquired consecutively by the acquisition device.

13. The system of claim 1, wherein the data D_{optical_flow_torso} is informative of three-dimensional optical flow between at least part of the torso of the user in the first image and at least part of the torso of the user in the second image, wherein the system is configured to generate data D_{optical_flow_torso_normalized} based on a transformation of the data D_{optical_flow_torso} into a volume of fixed size, and feed the data D_{optical_flow_torso_normalized} to the at least one machine learning model, to obtain the data D_{breathing} informative of the user's breathing.

14. The system of claim 1, wherein at least one of (i), (ii) or (iii) is met:
(i) the system is configured to obtain the data D_{breathing} informative of the user's breathing without requiring the user to take part to a calibration process;
(ii) the system is configured to determine the data D_{breathing} informative of the user's breathing in a scenario in which a position of the user is moving between the first image and the second image;
(iii) the system is configured to obtain data informative of the user's breathing for at least one given image of the sequence, even if the user was absent in one or more images of the sequence preceding this given image, without requiring the user to take part to a calibration process.

15. The system of claim 1, configured to perform at least one of (i), (ii), (iii) or (iv):
(i) detecting whether a frequency of change of the data D_{optical_flow_torso} in a plurality of images of the sequence is above a threshold, or
(ii) normalizing the data D_{optical_flow_torso} into normalized data D_{optical_flow_torso_normalized} and detecting whether a frequency of change of the data D_{optical_flow_torso_normalized} in a plurality of images of the sequence is above a threshold;
(iii) using the data D_{optical_flow_torso} to detect one or more occlusions between the user and an acquisition device which has acquired the sequence of images;
(iv) normalizing the data D_{optical_flow_torso} into normalized data D_{optical_flow_torso_normalized} and using the data D_{optical_flow_torso_normalized} to detect one or more occlusions between the user and an acquisition device which has acquired the sequence of images.

16. The system of claim 1, configured to feed the data D_{optical_flow_torso}, or the data derived therefrom, to the at least one machine learning model, to obtain data informative of the user's heart rate.

17. The system of claim 16, wherein the data informative of the user's heart rate includes at least one of:
(i) the user's heart rate, or
(ii) the user's heart rate variability.

18. The system of claim 1, configured to perform at least one of (i) or (ii):
(i) obtain data informative of the user's heart rate, and
feed the data D_{optical_flow_torso}, or data derived therefrom, and the data informative of the user's heart rate to the at least one machine learning model, to obtain data D_{breathing} informative of the user's breathing, or
(ii) obtain data informative of the user's heart rate,
obtain the data D_{breathing} informative of the user's breathing,
feed the data informative of the user's heart rate, or data derived therefrom, and the data D_{breathing} informative of the user's breathing to another machine learning model, to obtain at least one of:
updated data informative of the user's heart rate, or
updated data D_{breathing} informative of the user's breathing.

19. A non-transitory computer readable medium comprising instructions that, when executed by one or more processing circuitries, cause the one or more processing circuitries to perform:
obtaining a sequence of images of a user, the sequence of images comprising a first image and a second image,
determining data D_{optical_flow_torso} informative of a motion of at least part of a torso of the user between the first image and the second image, and
feeding the data D_{optical_flow_torso}, or data derived from the data D_{optical_flow_torso}, to at least one machine learning model, to obtain data D_{breathing} informative of the user's breathing.

20. A non-transitory computer readable medium comprising instructions that, when executed by one or more processing circuitries, cause the one or more processing circuitries to perform:
obtaining a sequence of images of a user, the sequence of images comprising a first image and a second image,
determining data D_{optical_flow_torso} informative of a motion of at least part of a torso of the user between the first image and the second image, and
feeding the data D_{optical_flow_torso}, or data derived from the data D_{optical_flow_torso}, to at least one machine learning model, to obtain data informative of the user's heart rate.
